(19) United States
(12) Patent Application Publication
(10) Pub. No.: US 2004/0260188 A1
(43) Pub. Date: Dec. 23, 2004

(54) Automated auscultation system

(75) Inventors: Zeeshan Hassan Syed, Wayzata, MN (US); John Guttag, Lexington, MA (US); Robert A. Levine, Brookline, MA (US); Francesca Nesta, Boston, MA (US); Dorothy Curtis, Framingham, MA (US)

Correspondence Address: Choate, Hall & Stewart, Exchange Place, 53 State Street, Boston, MA, 02109, US

(73) Assignees: The General Hospital Corporation; Massachusetts Institute of Technology

(21) Appl. No.: 10/464,267

(22) Filed: Jun. 17, 2003

(51) Int. Cl. A61B 5/04

(52) U.S. Cl. 600/509; 600/513; 600/528

(57) ABSTRACT

The present invention provides systems and methods for performing automated auscultation and diagnosis of conditions of the cardiovascular system. The invention acquires an acoustic signal emanating from the cardiovascular system via an sensor. In addition, in certain embodiments of the invention an electrical signal, e.g., an electrocardiogram (EKG) is simultaneously acquired. The signals are digitized, and, optionally, filtered to remove noise. The invention then processes and analyses the signal(s) so as to provide a clinically relevant conclusion or recommendation such as a diagnosis or suggested additional tests or therapy. In preferred embodiments the system comprises: (1) a beat selection component that selects a plurality of beats for analysis, wherein each beat comprises an acoustic signal emanating from the cardiovascular system; (2) a time-frequency analysis component that performs a time-frequency decomposition of beats selected for analysis so as to identify or extract physiologically relevant features; and (3) a processing component that processes the information so at to provide a clinically relevant conclusion or recommendation. In preferred embodiments of the invention the system further includes an aggregation component that combines information obtained from a plurality of the beats selected for analysis. In one embodiment, the system diagnoses mitral valve prolapse.

### GOVERNMENT SUPPORT

The United States Government has provided grant support utilized in the development of the present invention. In particular, Cooperative Agreement Number DAMD17-02-2-0006 from the Dept. of Defense has supported development of this invention. The United States Government may have certain rights in the invention.

### BACKGROUND OF THE INVENTION

Heart auscultation, the process of interpreting the sounds produced by the heart, is a fundamental tool in the diagnosis of diseases and conditions of the cardiovascular (CV) system. It serves as the most commonly employed technique for diagnosis of such diseases and conditions in primary health care and in circumstances where sophisticated medical equipment is not available, such as remote areas or developing countries. However, detecting relevant symptoms and forming a diagnosis based on sounds heard through a stethoscope is a skill that can take years to acquire and refine. Part of this difficulty stems from the fact that heart sounds are often separated from one another by very short periods of time [1]. In addition, the signals characterizing cardiac disorders are often less audible than normal heart sounds. This makes the task of acoustically detecting abnormal activity a challenge.

Even once the ability to perform auscultation is acquired, imparting it to others is a difficult task. The percentage of graduate medical training programs that incorporate structured teaching of auscultation is only 27.1% for internal medicine and 37.1% for cardiology [2]. This constitutes a further challenge to learning how to listen to heart sounds.

The decline in the practice and teaching of the diagnostic skill of cardiac auscultation has contributed to a situation in which both clinicians and physicians in training are quite inaccurate in the recognition of common auscultatory events [3] and in which there is increasing reliance on alternative diagnostic methods. Many of the contemporary “gold standard” tests for a variety of significant cardiac diseases that may also be diagnosed using auscultation are expensive and often unnecessary. In fact, as many as 80 percent of patients referred to cardiologists have only benign heart murmurs or normal hearts [ 1, 3, 4]. These cases represent a severe inefficiency as far as medical care is concerned, since the cost of a visit to a cardiologist (including associated echocardiography) runs anywhere from $300 to $1000 in the United States [1]. Such false positives also constitute a significant waste of time for both patients and cardiologists and are also the source of much unnecessary emotional anxiety for patients and their families. In addition to this, there are many forms of heart disease that remain asymptomatic, and thereby undetected, for several years until they eventually progress into serious medical disorders.

Accordingly, there is a need in the art for systems and methods that would assist clinicians in the use of auscultation for diagnosis and evaluation of cardiovascular conditions. Furthermore, there is a need for systems and methods that would allow the benefits of auscultation to be obtained with a reduced learning curve, using equipment that is low-cost, robust and easy to use. There is also a need for systems that would assist in teaching and acquiring the skills of auscultation. In addition there is a need for systems and methods that would improve on the accuracy of currently available tools and methods for performing auscultation.

### SUMMARY OF THE INVENTION

The present invention addresses the foregoing needs and others. The invention provides a system for performing automated auscultation. By “automated auscultation” is meant that the system performs a software or hardware based analysis of an acoustic signal or signals emanating from the cardiovascular system and, optionally an electric signal (EKG) emanating from the cardiovascular system and/or an acoustic signal emanating from the respiratory system. The system may further provide a conclusion or recommendation based on the signals. In a fully automated system, the only role(s) played by the user are in acquisition of the signals (e.g., placing sensors on the subject, controlling the devices that acquire the signal, etc.) and, optionally, providing information related to the conditions under which the signals were acquired (e.g., patient position) or clinical information to the system. In a system that is less than fully automated, users may play a variety of roles such as selection of beats for analysis, interpreting the information generated by the system in order to arrive at a conclusion or recommendation, etc. In certain preferred embodiments of the present invention the system is fully automated, e.g., it does not rely on the human ear and/or eye to select beats, to arrive at a conclusion or recommendation, etc.

Although in certain embodiments of the invention a conclusion or recommendation is provided by the system, it is noted that the systems and methods of the invention are also of use in contexts in which the user interprets the output of the system to arrive at a conclusion or recommendation. For example, the automated auscultation system of the invention provides a variety of novel ways of analyzing and presenting acoustic and/or electrical signals emanating from the cardiovascular system. Individuals such as health care providers or others may utilize the output of the inventive system to arrive at a clinical conclusion or recommendation. The system itself may or may not provide a conclusion or recommendation.

The automated auscultation system of the invention may be used to perform automated auscultation of the cardiovascular system, the respiratory system, or both. The invention may be used to assist a clinician in performing a number of different tasks including but not limited to: differentiating pathological from benign heart murmurs, detecting cardiovascular diseases or conditions that might otherwise escape attention, deciding whether to refer a patient for a diagnostic study such as an echocardiography or to a specialist, monitoring the course of a disease and the effects of therapy (which may include comparing current data for a subject to past data for the same subject), deciding when additional therapy or intervention is necessary, and providing a more objective basis for the decision(s) made. According to certain embodiments of the invention the system functions in a transparent manner that makes it easier to validate its performance in light of physiological knowledge. This approach may thereby increase clinician acceptance and facilitate communication of the ability to perform auscultation to others.

According to certain preferred embodiments of the invention the system detects and utilizes information obtained from a plurality of signals emanating from one or more organs of the cardiovascular system (i.e., heart and blood vessels, which are considered organs for purposes of the present invention) and/or the respiratory system (i.e., lungs and respiratory passages such as bronchi). According to certain embodiments of the invention the plurality of signals emanate from a single unitary component of the cardiovascular system, e.g., the heart. According to other embodiments of the invention the plurality of signals emanate from each of two individual, substantially symmetric components of the cardiovascular system, such as the corresponding right and left carotid, renal, femoral arteries, etc.

According to certain embodiments of the invention the system detects and utilizes signals that reflect different forms of energy. For example, the system may detect and utilize information obtained from an audio signal and an electrical signal. The audio and electrical signals may emanate from a single organ such as the heart, or from two or more different organs such as the heart and a lung, the heart and a blood vessel, etc. The audio and electrical signals may also emanate from two or more portions of a single organ, such as the atria, ventricles, and/or valves of the heart, etc. According to certain embodiments of the invention the system detects and utilizes information obtained from two audio signals that emanate from each of two individual, substantially symmetric components of the cardiovascular system such as two paired blood vessels. According to yet other embodiments of the invention the system detects and utilizes information obtained from acoustic signals that emanate from a plurality of individual components of the cardiovascular system (e.g., blood vessels or organs), which may or may not be symmetric. According to certain embodiments of the invention the system detects and utilizes information obtained from an audio signal emanating from one organ and an electrical signal emanating from a different organ.

In certain embodiments of the invention the signals are correlated, either in that each of them reflects the same underlying physiological event, or in that one of the signals causes or triggers a physiological event that gives rise to one or more of the other signal(s). For example, the electrical activity of the heart and the heart sounds are correlated in that the electrical activity is responsible for the muscle contractions that give rise to closure of heart valves and movement of blood that are responsible for the audio signals that are heard during clinical auscultation. The audio signals emanating from the two paired carotid arteries following each heartbeat are correlated in the sense that both of them reflect the movement of blood through the vessel as a result of that heartbeat. According to certain embodiments of the invention the system integrates the information obtained from the plurality of signals in a manner that facilitates interpretation of the signals. As but one example, the invention may utilize information obtained from an electrical signal to identify components or features of an audio signal or to isolate a region of interest in the signal. As another example, the invention may compare the audio signals obtained from two paired vessels to identify features indicative of a disease or clinical condition in one of the vessels.

In certain embodiments of the invention the system detects situations in which signals are expected to correlate, but do not, and/or situations that are not expected to correlate but do. The system utilizes this information in the course of arriving at a clinical conclusion or recommendation.

The invention further provides associated methods for use of the system. In one aspect, the invention provides a method for diagnosis and evaluation of cardiovascular conditions characterized by heart murmurs, e.g., valvular conditions such as mitral valve prolapse (MVP). In another aspect, the invention provides a method for diagnosis of cardiovascular conditions characterized by abnormally restricted blood flow in one or more vessels, e.g., arterial stenosis.

In one aspect, the invention provides a method of performing automated auscultation of the cardiovascular system, the method comprising steps of: (a) selecting one or more beats for analysis, wherein each beat comprises an acoustic signal emanating from the cardiovascular system; (b) performing a time-frequency analysis of beats selected for analysis so as to provide information regarding the distribution of energy, the relative distribution of energy, or both, over different frequency ranges at one or more points in the cardiac cycle; and (c) processing the information to reach a clinically relevant conclusion or recommendation; and (d) presenting some or all of the information in a manner that facilitates a mechanistically comprehensible explanation of the basis for the conclusion or recommendation or how the conclusion or recommendation was reached. The method may further comprise providing a mechanistically comprehensible explanation of the basis for the conclusion or recommendation or of how the conclusion or recommendation was reached and/or presenting information using any of a number of audio-visual aids. In certain embodiments of the invention the step of selecting one or more beats for analysis is performed automatically.

In another aspect, the invention provides a method of performing automated auscultation of the cardiovascular system of a subject, the method comprising steps of: (a) selecting one or more beats for analysis, wherein each beat comprises an acoustic signal emanating from the cardiovascular system; (b) performing a time-frequency analysis of beats selected for analysis so as to provide information regarding the distribution of energy, the relative distribution of energy, or both, over different frequency ranges at one or more points in the cardiac cycle; (c) if more than one beat was selected in step (a), combining information obtained from a plurality of the beats selected for analysis; and (d) processing the information to reach a clinically relevant conclusion or recommendation.

In another aspect, the invention provides a method of performing automated auscultation of the cardiovascular system, the method comprising steps of: (a) selecting a plurality of beats; (b) constructing a prototypical beat by combining information from the plurality of beats; (c) computing a metric that characterizes the prototypical beat; and (d) classifying the prototypical beat as indicative of the presence or absence of a disease or condition, or of its severity, by comparing the metric computed in step (c) with a value for the metric characteristic of a normal subject.

In another aspect, the invention provides a method of performing automated auscultation of the cardiovascular system, the method comprising steps of: (a) selecting a plurality of beats; (b) constructing a prototypical beat by combining information from the plurality of beats; and (c) presenting the prototypical beat, a time-frequency decomposition of the prototypical beat, or both, to a user, wherein presenting the prototypical beat comprises displaying an image of the beat, playing a recording of the beat or an enhanced version thereof, or both.

In another aspect, the invention provides a method of performing automated auscultation of the cardiovascular system, the method comprising steps of: (a) selecting one or more beats for analysis, wherein each beat comprises an acoustic signal emanating from the cardiovascular system; (b) performing a time-frequency analysis of beats selected for analysis so as to provide information regarding the distribution of energy, the relative distribution of energy, or both, over different frequency ranges at one or more points in the cardiac cycle; and (c) presenting information derived at least in part from the acoustic signal, wherein the information comprises one or more items selected from the group consisting of: a visual or audio presentation of a prototypical beat, a display of the time-frequency decomposition of one or more beats or prototypical beats, and a playback of the acoustic signal at a reduced rate with preservation of frequency content, wherein any of the foregoing items may be annotated or unannotated.

The invention further provides systems for performing the above methods and others. In certain embodiments of the invention the systems comprise a computer, a sensor, and/or an electronic stethoscope including a sensor.

In another aspect, the invention provides a system for assisting in the clinical evaluation of a subject's cardiovascular system comprising: an apparatus for performing automated auscultation of the cardiovascular system of a subject, wherein the apparatus analyzes an acoustic signal emanating from the cardiovascular system and provides a clinically relevant conclusion or recommendation; and at least one audio-visual diagnostic aid that presents information derived at least in part from the acoustic signal.

In another aspect, the invention provides a system for assisting in the clinical evaluation of a subject's cardiovascular system comprising: an apparatus for performing automated auscultation of the cardiovascular system of a subject; and at least one audio-visual diagnostic aid that presents information derived at least in part from the acoustic signal, wherein the information comprises one or more items selected from the group consisting of: a visual or audio presentation of a prototypical beat, a display of the time-frequency decomposition of one or more beats or prototypical beats, and a playback of the acoustic signal at a reduced rate with preservation of frequency content, wherein any of the foregoing items may be annotated or unannotated.

In another aspect, the invention provides methods and systems for automatic identification of the second heart sound (S2).

In another aspect, the invention provides methods and systems for construction of a prototypical beat, a frequency decomposition of a prototypical beat, audio-enhanced versions of the prototypical beat, and visual representations of the prototypical beat and its frequency components. The prototypical beat is useful in practicing the automated auscultation methods of the invention and also for a variety of other purposes.

In another aspect the invention provides methods and systems for evaluation of a subject with respect to mitral valve prolapse, e.g., for determining whether the subject suffers from mitral valve prolapse. The method performs a time-frequency analysis of an acoustic signal emanating from the subject's cardiovascular system and examines the energy content of the signal in one or more frequency bands, particularly higher frequency bands, in order to determine whether a subject suffers from mitral valve prolapse.

In another aspect, the invention provides an automated auscultation system augmented by a variety of audio-visual aids integrated with the system. According to certain embodiments of the invention the automated auscultation system provides a conclusion or recommendation. According to other embodiments of the invention the system provides information that assists a user in arriving at a conclusion or recommendation.

The contents of all papers, books, patents, web sites (as of Jun. 17, 2003) and other references, mentioned in this application are incorporated herein by reference.

### BRIEF DESCRIPTION OF THE DRAWINGS

FIG. 1 is a schematic diagram of a longitudinal section through the heart.

FIG. 2 is a schematic diagram of the cardiac cycle.

FIG. 3 is a more detailed schematic diagram of the cardiac cycle.

FIG. 4 is a schematic diagram of a sample EKG tracing.

FIG. 5 is a sample phonocardiogram tracing.

FIG. 6 is a sample phonocardiogram and EKG tracing, showing the occurrence of heart sounds in the cardiac cycle.

FIG. 7 is diagram showing certain auscultation sites in the human body.

FIG. 8 shows a variety of murmur patterns having different timing and morphology.

FIG. 9 is a schematic diagram of the heart of a subject suffering from mitral valve prolapse with regurgitation.

FIG. 10 shows an echocardiogram of a subject suffering from mitral valve prolapse.

FIG. 11 shows a sub-problem decomposition of automated auscultation according to the invention.

FIG. 12 shows simultaneously recorded acoustic and EKG signals.

FIG. 13 shows a block diagram of a system to extract systolic segments with beat selection.

FIG. 14 shows an example of a noisy beat discarded by the inventive system.

FIG. 15 shows a block diagram representation of a filter bank of the invention.

FIG. 16 shows a time-frequency decomposition illustrating the variation in amplitude of different frequency bands for the filter bank depicted in FIG. 15.

FIG. 17 shows a time-frequency decomposition corresponding to a filter bank using Hamming windows of length 50001 (transition band width of 3.5 Hz) for a subject suffering from moderate MVP with late systolic regurgitation.

FIG. 18 shows a time-frequency decomposition corresponding to a filter bank using Hamming windows of length 1001 (transition band width of 176.2 Hz) for the same subject as in FIG. 17.

FIG. 19 shows the unaggregated output of the filter bank of FIG. 15, for the same subject as in FIG. 17 (16 output bands).

FIG. 20 shows the output of the filter bank of FIG. 15 with aggregation of all frequencies above 100 Hz into one band (2 output bands).

FIG. 21 is a block diagram representation of the filter bank of FIG. 15, with band aggregation.

FIG. 22 shows the output of the filter bank of FIG. 15 with post-normalization aggregation of frequencies above 100 Hz into one band (2 output bands).

FIG. 23 shows the output of the filter bank of FIG. 21 with post-normalization aggregation of frequencies into four different bands (4 output bands) for the same subject as in FIG. 17.

FIG. 24 is a block diagram representation of a filter bank with band aggregation and time-envelope characterization.

FIG. 25 shows the output of the filter bank of FIG. 20 with time-envelope characterization and post-normalization aggregation of frequencies into four different bands (4 output bands) for the same acoustic recording as in FIG. 24.

FIG. 26 shows an example prototypical beat calculation.

FIG. 27 shows a time-frequency decomposition for a non-MVP subject.

FIG. 28 shows a time-frequency decomposition for a second non-MVP subject.

FIG. 29 shows a time-frequency decomposition for a third non-MVP subject.

FIG. 30 shows a time-frequency decomposition for an MVP subject with high frequency peaks shifted significantly prior to S2.

FIG. 31 shows a time-frequency decomposition for a second MVP subject with high frequency peaks shifted significantly prior to S2.

FIG. 32 shows a time-frequency decomposition for a third MVP subject with high frequency peaks shifted significantly prior to S2.

FIG. 33 shows a time-frequency decomposition for an MVP patient with wider high frequency peaks extending into systole.

FIG. 34 shows a time-frequency decomposition for a second MVP patient with wider high frequency peaks extending into systole.

FIG. 35 shows a time-frequency decomposition for a third MVP patient with wider frequency peaks extending into systole.

FIG. 36 shows a time-frequency decomposition for an MVP patient with additional high frequency peaks.

FIG. 37 shows a time-frequency decomposition for a second MVP patient with additional high frequency peaks.

FIG. 38 shows a time-frequency decomposition for a third MVP patient with additional high frequency peaks.

FIG. 39 shows a comparison of the performance of the inventive system with the performance of primary care physicians.

FIG. 40 shows the sensitivity and specificity of the 150-350 Hz threshold value for diagnosis of MVP.

FIG. 41 shows the sensitivity and specificity of the 350-550 Hz threshold value for diagnosis of MVP.

FIG. 42 shows the sensitivity and specificity of the 550-850 Hz threshold value for diagnosis of MVP.

FIG. 43 shows a time-frequency visualization of a sample beat corresponding to an MVP patient.

FIG. 44 shows a time-frequency visualization of a prototypical beat for a non-MVP patient.

FIG. 45 shows a time-frequency visualization of a prototypical beat for an MVP patient.

FIG. 46 shows a representative embodiment of a computer system and electronic stethoscope for use in the context of the present invention.

### DETAILED DESCRIPTION OF CERTAIN EMBODIMENTS

I. Overview of the Invention

This section provides a brief overview of the invention according to certain preferred embodiments. The invention acquires an acoustic signal emanating from the cardiovascular system via a sensor. In addition, in certain embodiments of the invention an electrical signal, e.g., an electrocardiogram (EKG) is simultaneously acquired. The signals are digitized, and, optionally, filtered to remove noise. The invention then processes and analyses the signal(s) so as to provide a clinically relevant conclusion or recommendation such as a diagnosis or suggested additional tests or therapy. The invention thus assists the user (e.g., physician, nurse, or any other health care provider, or any other individual using the system) in deciding upon the appropriate course to take when screening or evaluating a patient for conditions of the cardiovascular system, (e.g., whether to refer the patient for additional tests or to a specialist, whether to initiate therapy, etc.) The systems and methods may be applied to either adult or pediatric subjects and may be used to provide diagnostic support and/or as a screening tool. For example, the system may be employed as part of the evaluation of newborn infants and added to the standard tests that are performed to yield an Apgar score. The system may be used in situations in which no professional health care provider is involved, e.g., as part of a home health care activity. 100751 According to certain embodiments of the system a variety of audio-visual aids are included. These aids support the clinical conclusion or recommendation made by the system. Certain of the aids display processed acoustic signals in a way that reveals the basis on which the system arrived at a conclusion, so as to enhance confidence in the system and help make its operation transparent to the use. These features and others make the aids useful for teaching purposes and by practitioners to enhance their understanding of the pathophysiology of the cardiovascular system and the way in which clinical conditions are reflected in alterations in the acoustic signals emanating from the heart and/or blood vessels.

In preferred embodiments the system comprises: (1) a beat selection component that selects a plurality of beats for analysis, wherein each beat comprises an acoustic signal emanating from the cardiovascular system; (2) a time-frequency analysis component that performs a time-frequency decomposition of beats selected for analysis so as to identify or extract physiologically relevant features; and (3) a processing component that processes the information so at to provide a clinically relevant conclusion or recommendation. In preferred embodiments of the invention the system further includes an aggregation component that combines information obtained from a plurality of the beats selected for analysis. Preferred embodiments of the invention are fully automated, i.e., they do not require user intervention for beat selection or for any of the other analytic or diagnostic steps (other than acquiring the signals, which involves applying a sensor such as that contained in an electronic stethoscope and EKG leads to the subject's body).

The components employ a number of different methods for performing their tasks. In particular, in certain embodiments of the invention the beat selection component implements a method for segmenting the acoustic signal that involves locating the second heart sound (S2). In preferred embodiments of the invention time-frequency decomposition is performed by filtering the acoustic signal with a bank of filters having different passbands, and the analysis involves combining the outputs of these filters into broader, aggregated bands. In preferred embodiments of the invention, instead of, or in addition to, analyzing individual beats, the methods involve construction of a prototypical beat by aggregating information from multiple individual beats. Such aggregation may be performed either before or, in preferred embodiments, after decomposing the beats into frequency components.

It is noted that each of the components mentioned above was developed without requiring use of machine learning (e.g., neural networks) or employing a purely statistical approach to recognition of significant features, e.g., features such as normal heart sounds and/or sounds indicative of abnormality. Avoidance of such methods makes it easier to validate the system in light of physiological knowledge and makes it more possible to allow a typical user to understand the basis on which the invention arrives at a clinical conclusion or recommendation. The approach taken by the invention, by emphasizing human understanding of physiological and pathological events, allows the system to present information in a manner that facilitates a mechanistically comprehensible explanation of the basis for a conclusion or recommendation reached by the system, or how the conclusion or recommendation was reached. In certain embodiments of the invention the system provides such a conclusion. In addition, this information is useful even in those embodiments of the invention in which the system does not provide a conclusion or recommendation in that it is interpretable by a clinician and assists the clinician in arriving at a conclusion or recommendation.

Furthermore, in preferred embodiments of the invention the system is implemented without requiring additional equipment beyond a commercially available electronic stethoscope with EKG leads and a personal computer and without requiring additional maneuvers beyond those that would be performed in a typical physical examination.

It is noted that although the invention is described in terms of signals acquired noninvasively, i.e., from the exterior of the body, the inventive system and methods may also be employed in the context of signals acquired from within the body. For example, sensors may be implanted inside the body either temporarily or permanently (e.g., as part of a device such as a pacemaker or implantable defibrillator). Specific parameters and other details of the algorithms used to process and analyze the signals may differ when signals are acquired from within the body.

According to preferred embodiments the invention is implemented as a software application system, which may be embodied on a computer-readable medium or storage device. However, it will be appreciated that the invention may be implemented in various forms of hardware, software, firmware, special purpose processors, or combinations of any of these.

The next section provides a description of the physiology of the heart and of heart murmurs to facilitate understanding of the invention. The following sections describe the operation of the various components and methods mentioned above. The description focuses on a preferred embodiment of the invention that diagnoses mitral valve prolapse, a cardiac condition frequently encountered in clinical practice, and one that poses significant diagnostic challenges. However, it is to be understood that the systems and methods of the invention are applicable to a wide range of other conditions of the cardiovascular system that are characterized by abnormalities in the acoustic signal emanating from the heart and/or blood vessels. These conditions include valvular disorders (e.g., regurgitation, stenosis, or sclerosis of the mitral, aortic, pulmonic, and/or tricuspid valves, including disorders of artificial valves), vascular stenosis (e.g., carotid stenosis, renal stenosis, femoral stenosis, or stenosis of any of the more peripheral arteries), patent ductus arteriosus, septal defects, etc. The system may diagnose such conditions, distinguish between them (e.g., distinguish between aortic stenosis and sclerosis or between different valvular pathologies, etc.), provide an indication of their severity, make therapeutic recommendations or recommend additional tests, etc. For purposes of description it is assumed that the system is used for evaluation of the heart, thus beats may be referred to as heart beats and the acoustic signal may be referred to as heart sounds herein.

When the system is used in the evaluation of conditions of the arteries such as stenosis or sclerosis, acoustic signals may be acquired from multiple arteries, e.g., right and left members of an arterial pair such as the carotid arteries. Alternately, acoustic signals may be acquired from arteries located more or less peripherally in relation to the heart. For example, signals may be acquired from the femoral artery and the popliteal or pedal artery for the diagnosis of peripheral vascular disease. Features of the acoustic signals acquired from multiple arteries may be compared. Differences between signals may be used to arrive at a conclusion or recommendation.

II. Cardiovascular Physiology and Heart Murmurs

(A) Cardiac Anatomy and the Cardiac Cycle

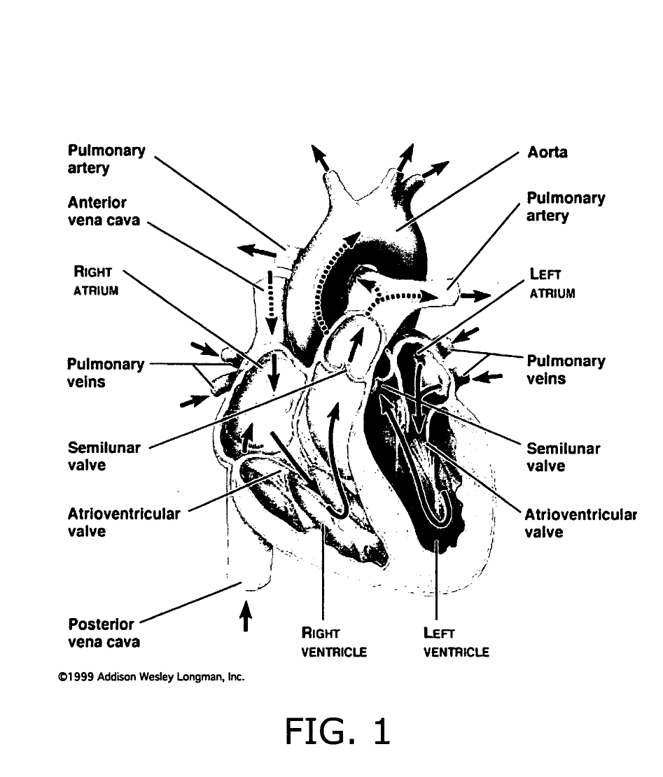

FIG. 1 provides a visual representation of the human heart. As depicted therein, the atria are separated from their respective ventricles by the atrioventricular (AV) valves, i.e., the tricuspid valve, which separates the right atrium from the right ventricle, and the biscuspid or mitral valve, which separates the left atrium from the left ventricle. The pulmonary valve separates the right ventricle from the pulmonary artery, while the aortic valve separates the left ventricle from the aorta. The latter two valves prevent the back flow of blood from the arteries into the ventricles.

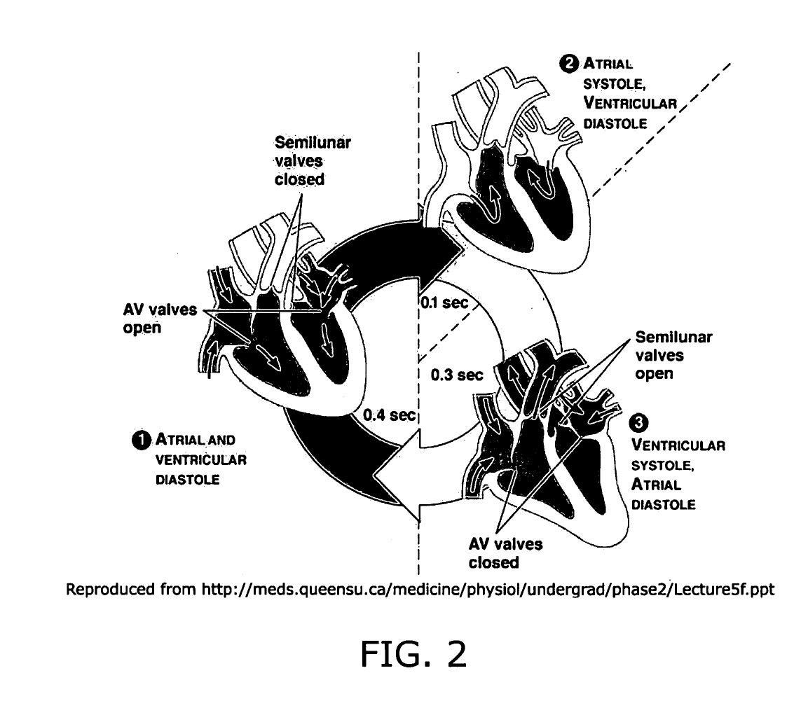

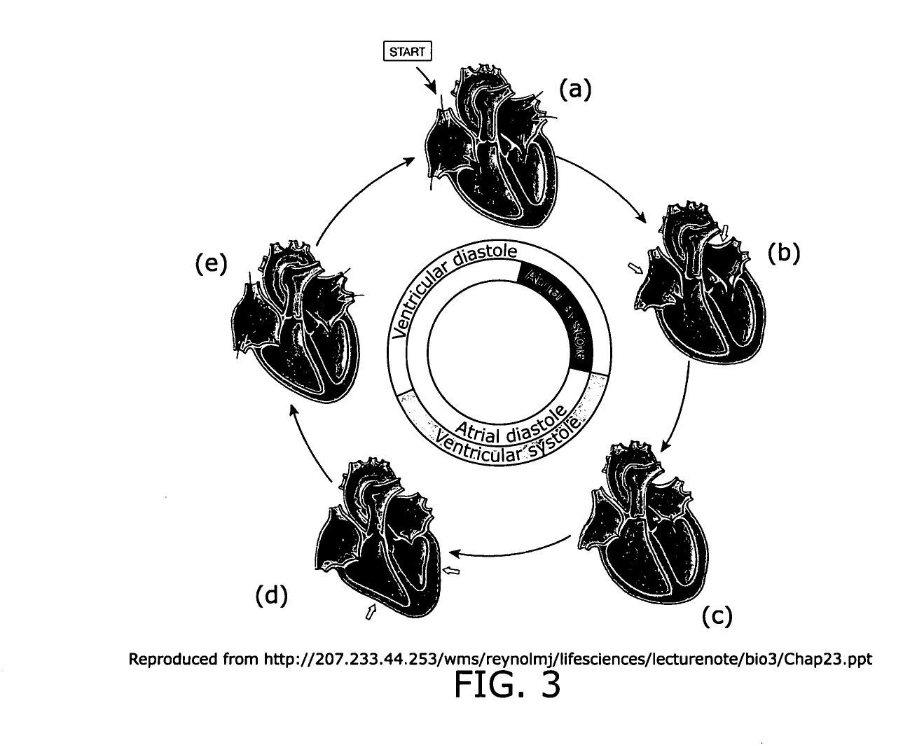

The periodic pumping action of the heart that results in the (normally) unidirectional flow of blood through the human body is known as the cardiac cycle (FIGS. 2 and 3). The heart rate and duration of each beat vary significantly between people and may have different values for the same individual, depending, e.g., on the activity being performed. The terms “beat” and “heart beat” are used interchangeably herein and are to be given their meaning as generally accepted in the art. In general, these terms refer to the electrical and mechanical events associated with contraction of the cardiac muscle during a single cardiac cycle, as well as the electrical and acoustic signals emanating from the cardiovascular system that reflect these events, regardless of where these signals are detected (e.g., acoustic signals associated with beats may emanate from blood vessels as well as from the heart). The electrical events include depolarization and repolarization of the conducting system and cardiac muscle (discussed below). The mechanical events include contraction of the cardiac muscle, blood flow, and closure and opening of the valves.

Heart beats are divided into systole and diastole, with systole being further divided into atrial and ventricular systole. During the latter, ventricular contraction raises the pressure in the ventricles, causing the AV valves to close once this pressure exceeds that in the atria. Ventricular pressure continues to rise, causing opening of the semilunar valves when it exceeds the pressure in the aorta and pulmonary artery, which allows blood to flow out of the ventricles. Ventricular pressure then decreases until eventually it falls below that in the arteries, causing blood in the arteries to start flowing back towards the ventricles, leading to closure of the semi-lunar valves. During this period the atria are filling with blood, causing pressure in them to rise until it exceeds that in the ventricles, which leads to opening of the AV valves. The cycle then repeats.

(B) Electrical Activity and the Electrocardiogram

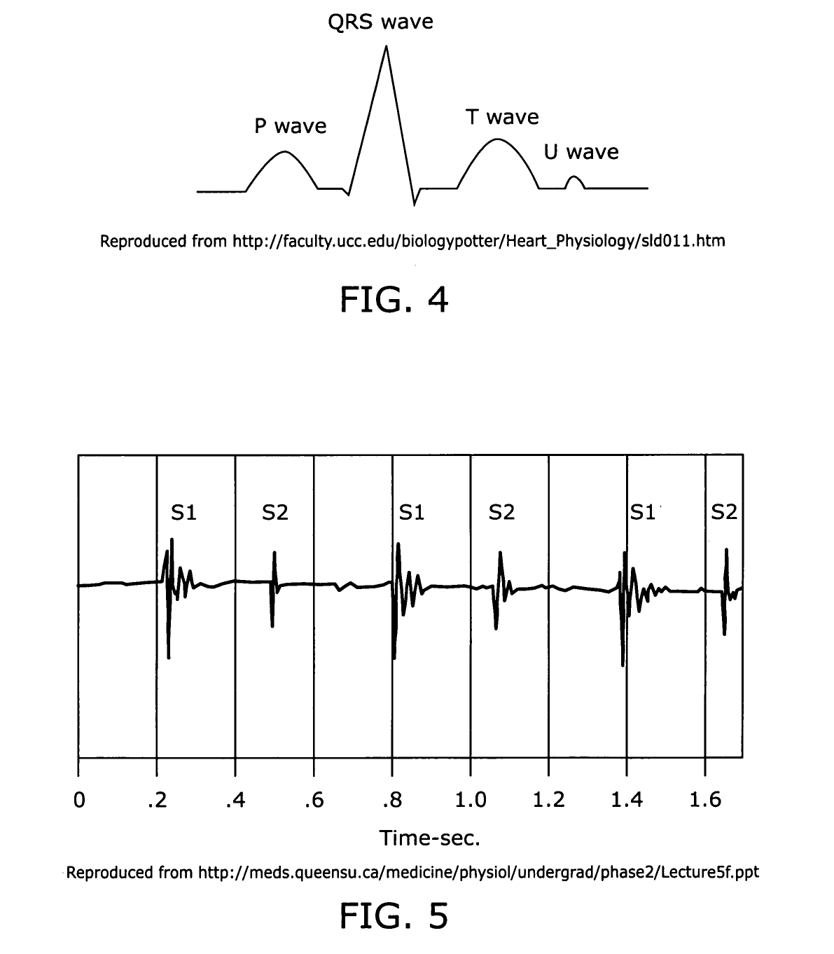

The electrocardiogram (EKG) is a record of the electrical activity occurring in the heart during one cardiac cycle. Cardiac electrical activity is generated and propagated by the conduction system, which comprises the sinoatrial (SA) node, atrioventricular (AV) node, AV bundle, bundle branches, and conduction myofibers. A representative sketch of a typical EKG is shown in FIG. 4. The EKG is composed of three (or sometimes four) distinct deflections or waves, with intervals between them. The P wave reflects depolarization of the atria as the action potential generated by the SA node travels downward through them, eventually reaching the AV node. Activation of the left bundle branch results in the small negative deflection referred to as the Q wave. Depolarization of the ventricles is responsible for the large upward deflection known as the R wave. Depolarization of the last portion of ventricular muscle leads to the negative S wave. Following the isoelectric ST segment, repolarization causes the T wave. The U wave (which is so small that it is often undetected) results from repolarization of the AV bundle. It is noted that the above description refers to a typical normal EKG. The pattern described above may vary, e.g., in the case of cardiac disease, etc.

(C) Normal Acoustical Activity

The phoncardiogram is a record of the acoustical activity occurring in the heart during one cardiac cycle. FIG. 5 depicts a sample phonocardiogram tracing. The most obvious of the sounds associated with normal function of the heart are the first and second heart sounds, S1 and S2 [54]. S1 marks the approximate beginning of systole and results from closing of the AV valves. It occurs slightly after the QRS complex in the EKG. S2 occurs at the end of systole as a result of closure of the AV valves. S2 occurs at the end of the T wave.

Although S1 and S2 are generally considered to be discrete sounds, each is generated by the near-simultaneous closing of two separate valves. For many purposes it is sufficient to consider each of these sounds as being single and instantaneous. However, certain conditions (including various benign and pathological conditions as well as physical maneuvers and physiological events) can split each heart sound into its separate components. Knowing the order of valve closure facilitates understanding the different reasons for the splitting of heart sounds. During S1, closing of the mitral valve slightly precedes closing of the tricuspid valve, while in S2 the aortic valve closes shortly before the pulmonary valve. Because diastole normally takes about twice as long as systole, there is a longer pause between S2 and S1 than between S1 and S2. However, rapid heart rates can shorten diastole to the point where it is difficult to discern which is S1 and which is S2. Information in the EKG can be used to make this determination.

Two additional sounds may occur during the cardiac cycle, typically in association with various pathological conditions, though occasionally in their absence. S3 is a third heart sound and is due to rapid passive ventricular filling. It occurs in a variety of conditions including dilated congestive heart failure, severe hypertension, myocardial infarction, or mitral incompetence. S4 is a fourth heart sound that is associated with atrial contraction against a stiffened ventricle. It is often associated with conditions such as aortic stenosis or hypertensive heart disease and may also occur in heart failure.

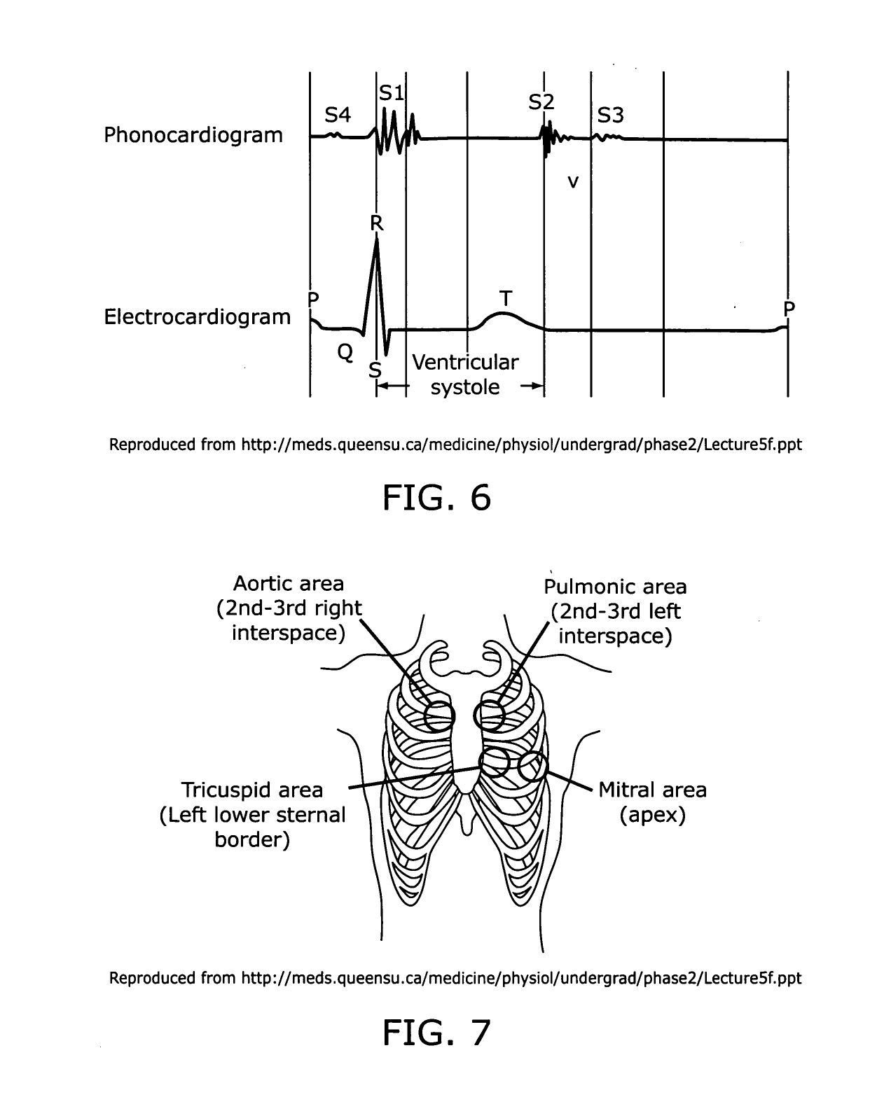

FIG. 6 illustrates the position of these heart sounds in the cardiac cycle. Generally, the sounds produced by each valve are best detected over a particular region of the chest, as shown in FIG. 7. Normally only S1 and S2 can be heard using a typical stethoscope. The S3 sound is often prominent in children, and in certain cases S4 can be distinguished in normal patients. Characteristics of the heart sounds, and their location relative to features of the EKG, as well as knowledge of the clinical conditions and diseases that may affect their timing, amplitude and/or frequency content are used in the present invention.

(D) Abnormal Acoustic Activity—Heart Murmurs

When a valve is damaged or stenotic, the abnormal turbulent flow of blood produces an audible “swooshing” sound known as a murmur [54, 9, 10]. (Turbulence in blood vessels, e.g., due to stenosis or physical abnormalities, may also produce abnormal sounds that may be used in the evaluation of these conditions.) Murmurs may be classified based on their timing, severity, location, shape and sound quality, conditions under which they may be more or less easily detected, clinical significance, etc.

Murmurs are generally distinguished as systolic and/or diastolic by timing them against S1 and S2. Murmurs that completely occupy systole are referred to as holosystolic. Murmurs with discrete start and end points are classified as early, mid, or late systolic, depending on the timing. Regurgitant murmurs, such as mitral valve insufficiency, often fill the entire phase, while ejection murmurs, such as aortic stenosis, usually have noticeable start and end points within that phase.

In the context of aural diagnosis, murmur intensity is frequently graded according to the Levine scale [11, 12]:

I—Lowest intensity, difficult to hear even by expert listeners

II—Low intensity, but usually audible to all listeners

III—Medium intensity, easy to hear even by inexperienced listeners, but without a palpable thrill

IV—Medium intensity with a palpable thrill

V—Loud intensity with a palpable thrill. Audible even with the stethoscope placed on the chest with the edge of the diaphragm.

VI—Loudest intensity with a palpable thrill. Audible even with the stethoscope raised above the chest.

A murmur may not be audible over all areas of the chest. The exact locations at which a murmur may be heard may vary according to the underlying pathology. Information regarding the location from which an acoustic signal was acquired is of use in the present invention.

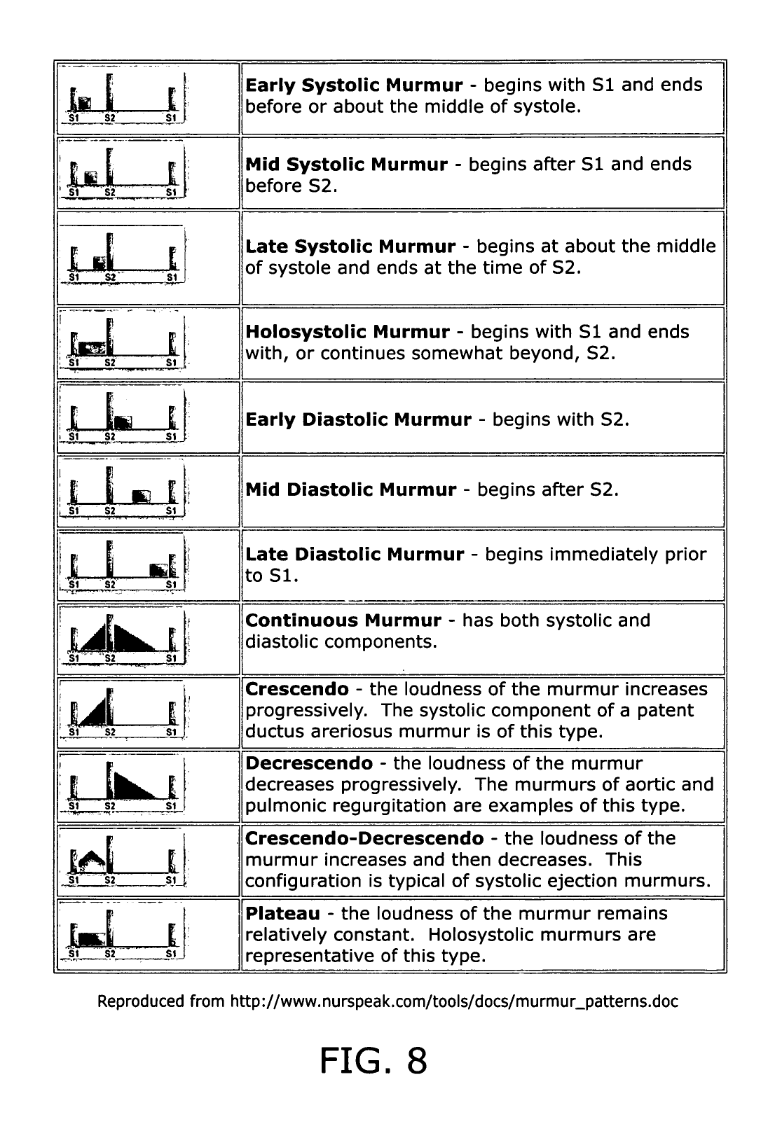

Murmurs may possess many different morphologies. The shape of the murmur may be continuous (uniform/constant), a plateau (constant through systole), a crescendo (increasing), a decrescendo/diminuendo (decreasing) or a crescendo-decrescendo (diamond-shaped murmur). Common descriptive terms for sound quality include rumbling, blowing, machinery, scratchy, harsh, or musical. FIG. 8 illustrates different murmur patterns based on timing and morphology. Murmurs associated with various conditions and diseases of the cardiovascular system typically have a characteristic timing and morphology. For example, aortic and pulmonic regurgitation are diastolic murmurs that display a decrescendo morphology. Information regarding the timing and morphology of murmurs is useful in the context of the present invention, e.g., for selecting regions of interest to examine within beats.

Dynamic maneuvers, e.g., placing the patient in particular positions such as lying or squatting while acquiring the acoustic signal, asking the patient to perform a Valsalva maneuver (which is often performed in the clinical diagnosis of heart abnormalities and is performed by attempting to forcibly exhale while keeping the mouth and nose closed), etc., may affect the signal, sometimes making it more easy to detect. In addition, the audibility of certain murmurs may be accentuated by inspiration and expiration.

Murmurs may or may not be clinically significant. This follows from the fact that whereas a murmur may be caused by normal blood flow through an impaired valve, it may also be created by high flow through a normal valve. Pregnancy is a common high-volume state where these physiologic flow murmurs are often heard. Anemia and thyrotoxicosis can also cause high-flow situations where the murmur is not pathologic itself, but indicates an underlying disease process. Children also frequently have innocent murmurs which are not due to underlying structural abnormalities.

(E) Mitral Valve Prolapse and Associated Murmurs

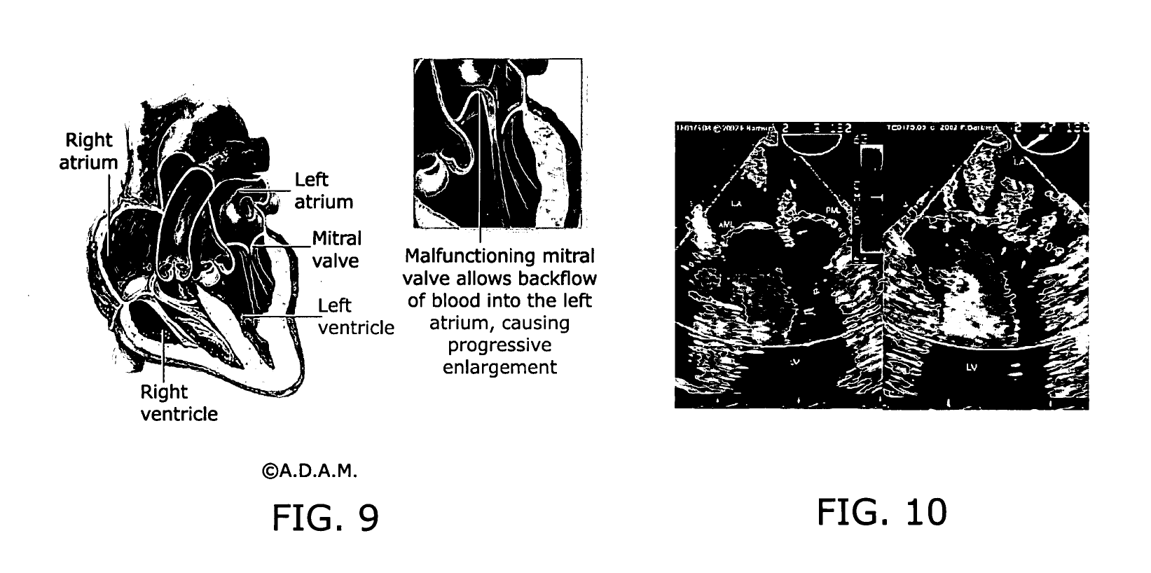

According to one embodiment of the invention, the system is used in the evaluation of mitral valve prolapse (MVP), a heart condition that is frequently diagnosed in healthy people and is usually harmless [5, 6, 7, 8]. The condition arises when the shape or dimensions of the leaflets of the mitral valve are not ideal, preventing them from closing properly and leading them to balloon out. The flapping of the leaflets may result in a clicking sound. In some cases, the prolapsing of the valve may allow a slight flow of blood back into the left atrium (mitral regurgitation), which gives rise to a murmur. FIG. 9 is a visual depiction of MVP.

Most individuals with MVP have no discomfort though some may report mild symptoms such as shortness of breath, dizziness and either skipping or racing of the heart. More rarely, chest pain is reported. However, these symptoms may not necessarily be related to MVP and as a result it is difficult to make a diagnosis based solely on whether or not a patient exhibits such behavior. Instead, diagnosis proceeds either by means of auscultation (whereby a doctor uses a stethoscope to listen to the sounds produced by the heart) or by means of an echocardiogram as shown in FIG. 10. Although an echocardiogram is the gold standard for evaluating the presence of MVP, it is relatively expensive, and this makes a strong case for promoting the use of auscultation to screen for and/or detect MVP.

In most cases, patients diagnosed as suffering from MVP require no special treatment. However, in the case of mitral regurgitation the flow of blood back into the left atrium causes an increased risk of acquiring bacterial endocarditis. To prevent this, many physicians and dentists prescribe antibiotics before certain surgical or dental procedures. Also, patients with significant mitral regurgitation generally need to be followed more closely by their physicians. In certain cases, surgical repair or valve replacement may be necessary if the condition worsens. In addition, anti-arrhythmics (drugs which regulate the heart rhythm) may be needed to control irregular heart rhythms. Vasodilators (drugs that dilate blood vessels) also help reduce the workload of the heart and digitalis may be used to strengthen the heart beat.

Table 1 details the evaluation and management of MVP disorders of increasing severity. The table classifies subjects based on risk category (risk of complications), recommended diagnostic studies, and recommended treatment (where the term “treatment” is taken to include prevention, prophylaxis, etc. In addition to determining whether or not a subject has MVP, the invention may also provide such classifications and recommendations. Similarly, the invention may provide appropriate classifications and recommendations for subjects suffering from other conditions or diseases of the cardiovascular system.

The murmurs associated with mitral valve prolapse are frequently somewhat complex. Following a normal S1 and an initial briefly quiet systole, the valve suddenly prolapses, resulting in a mid-systolic click. The click is characteristic of MVP and even without a subsequent murmur, its presence alone is enough for the diagnosis. Immediately after the click, a brief crescendo-decrescendo murmur occurs, which can be seen to peak during mid to late systole. This is usually heard best at the apex. The murmur is a result of the turbulent backflow of blood towards the end of systole. As the right ventricle contracts during systole, the pressure in this chamber continues to increase.

TABLE 1
Evaluation and Management of Mitral Valve Prolapse

| Risk Category | Echo Evaluation | Other Tests | Treatment |
|---|---|---|---|
| Low | Echocardiogram | Initial ECG | Education and |
| MVP without valvular | every 5 yr | 24-hr Holter | reassurance |
| deformity or | | monitor | For palpitations: beta- |
| regurgitation | | Graded exercise | blocker, dietary changes, |
| | | stress test | and regular exercise |
| Mild | Echocardiogram | Initial ECG | Oral antibiotic |
| MVP with valvular | every 2-3 yr | 24-hr Holter | prophylaxis |
| deformity and no | | monitor | Treat even mild |
| regurgitation | | Graded exercise | hypertension |
| | | stress test | Encourage weight loss if |
| | | Stress | needed |
| | | echocardiogram | Treat palpitations as |
| | | | above |
| Moderate | Echocardiogram | Initial EGG | Oral antibiotic |
| MVP with valvular | every 2-3 yr | 24-hr Holter | prophylaxis |
| deformity and mild | | monitor | Treat even mild |
| regurgitation | | Graded exercise | hypertension |
| | | stress test | Encourage weight loss if |
| | | Stress | needed |
| | | echocardiogram | Treat palpitations as |
| | | | above |
| High | Doppler | Initial ECG | As above and closely monitor |
| MVP with moderate- | echocardiogram | 24-hr Holter | cardiac function and replace |
| to-severe regurgitation | every yr | monitor | mitral valve when necessary |
| | | Graded exercise | |
| | | stress test | |
| | | Stress | |
| | | echocardiogram | |
| | | Others based on | |
| | | signs and | |
| | | symptoms | |

Eventually, it becomes sufficiently high to force open the damaged mitral valve, pushing blood back into the right atrium. This flow of blood through the small orifice between the flaps of the valve gives rise to a high frequency murmur just before S2. In contrast to most other murmurs, MVP is enhanced by Valsalva maneuvres and decreased by squatting. MVP murmurs are also heard better with patients lying down [14].

The presence of significant mitral regurgitation often leads to a holosystolic murmur. The mitral valve in such cases is compromised to an extent that it permits backflow of blood for the entire systolic period rather than simply towards the end of systole when the pressure in the ventricles is sufficiently high to force blood back into the atria. The quality of the murmur is usually described as blowing, and is often associated with an S3 because of the left atrial volume overload. Although S1 is due to a combination of mitral and tricuspid valve closure, the mitral valve is the louder aspect. Because the valve closure in mitral regurgitation is incomplete, S1 may be noticeably quieter. Finally, in severe regurgitation, the pressure in the left ventricle quickly equalizes with venous pressure in the left atrium during the start of diastole. The result is that the aortic valve may close prematurely and may occasionally result in a widely split S2.

Heart murmurs arising from MVP are best heard at the apex as shown in FIG. 7 and radiate into the axilla. Another useful site for detecting heart murmurs is the para-sternum or the fourth intercostal space. Information related to the timing of heart murmurs and the sites where they are most audible is useful in the context of the present invention, as described further below.

III. Methods and Components of the Automated Auscultation System

According to certain preferred embodiments of the invention the task of performing automated auscultation can be structured into a number of subproblems:

(1) beat selection to restrict analysis to a subset of the total beats recorded for the subject, with attention optionally focused on those beats that are determined to contain most diagnostic information based on a set of medically relevant criteria (e.g., beat length) and contain less noise;

(2) time-frequency analysis or decomposition of acoustic signals, which may be used to identify or extract physiologically significant features from the signals. Such features may include, for example, the presence of energy components in different frequency bands in a pattern that is correlated with a physiological event (e.g., closure of a valve, movement of blood through an opening in a valve);

(3) beat aggregation to combine information across multiple beats

(4) a decision mechanism that maps feature values to a clinical conclusion or recommendation. The decision mechanism may make use of a number of metrics and methods of classifying beats to arrive at the conclusion or recommendation.

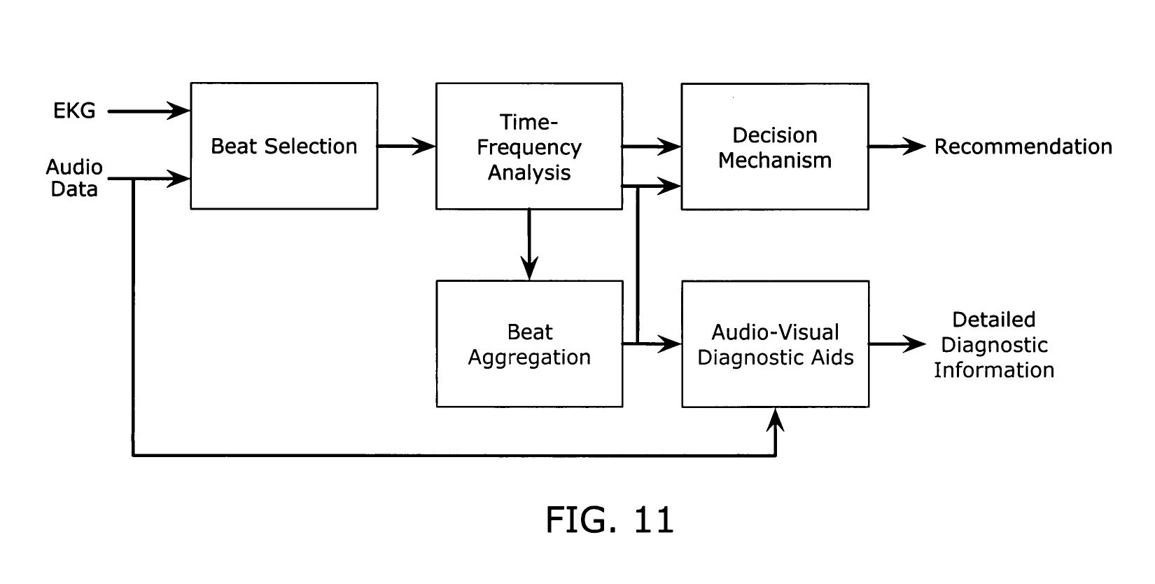

FIG. 11 presents a schematic showing decomposition of the task of automated auscultation into sub-problems, and their integration to arrive at an overall solution. Preferred embodiments of the invention also address the problem of providing audio-visual aids that may facilitate teaching and make the basis for the clinical conclusion or recommendation more evident to the health care provider. The components of the system are described in more detail below.

A. Signal Acquisition

The system acquires an acoustic signal emanating from the cardiovascular system and, optionally, an EKG signal. The signal may be acquired using a standard electronic stethoscope, comprising a sensor. In preferred embodiments of the invention the acoustic signal is acquired by placing the sensor on the surface of the patient's chest as is done in a typical examination of the cardiovascular system. The EKG may be acquired using a simple two-lead system (including a third lead as a reference) or, in certain embodiments of the invention, a one-lead system. The acquired acoustic and EKG signals are transferred to a computing device for processing and analysis of the digitized signals. The transfer step may be performed via physical electrical connections to the computing device or using a wireless interface. The signals are generally recorded and may be stored for future playback or display, for inclusion in a patient record, for transfer to another location, etc.

B. Beat Selection Component

In preferred embodiments of the invention the beat selection component uses the acoustic signal and the temporally aligned EKG signal to extract beats considered most likely to contain useful diagnostic information. For example, the beat selection component may reject beats deemed to contain too much noise and may selectively retain beats with increased diagnostic information based on a number of criteria, e.g., length of the beat.

(1) Segmentation

The beat selection component segments the acoustic signal into individual beats. In addition, in certain embodiments of the invention the beat selection component segments the acoustic signal into regions of interest, e.g., systolic region, diastolic regions, or portions of either of these regions. These regions may differ depending upon which clinical condition(s) are being examined. For example, murmurs associated with mitral valve prolapse occur primarily during systole, so it is appropriate in this case to focus on systolic regions. In general, as used herein the term “segmentation” refers to the separation of beats into regions of interest rather than separation of the acoustic signal into individual beats. For example, the term “segmentation” is used to refer to separating individual beats into systolic and diastolic regions. It is noted that separation of beats is performed implicity in locating S1 and S2 as described below.

Segmentation into systolic regions may be achieved by locating the QRS complex and S2 (with the interval between the two corresponding to systole). Prior to utilizing the EKG signal, it may be desirable to filter it, e.g., to remove baseline fluctuation and/or noise. For example, baseline fluctuation may be removed by excluding frequencies below approximately 1.5 Hz. High frequency noise can be addressed by considering only those frequencies below a predetermined number of Hz, e.g., 100 Hz. In band interference, e.g., a 60 Hz hum, may also be filtered out. Appropriate filtering can be performed using, for example, a finite impulse response approximation to the infinite impulse response of an ideal filter [20]. Any of a large number of other filter types may be used for this purpose.

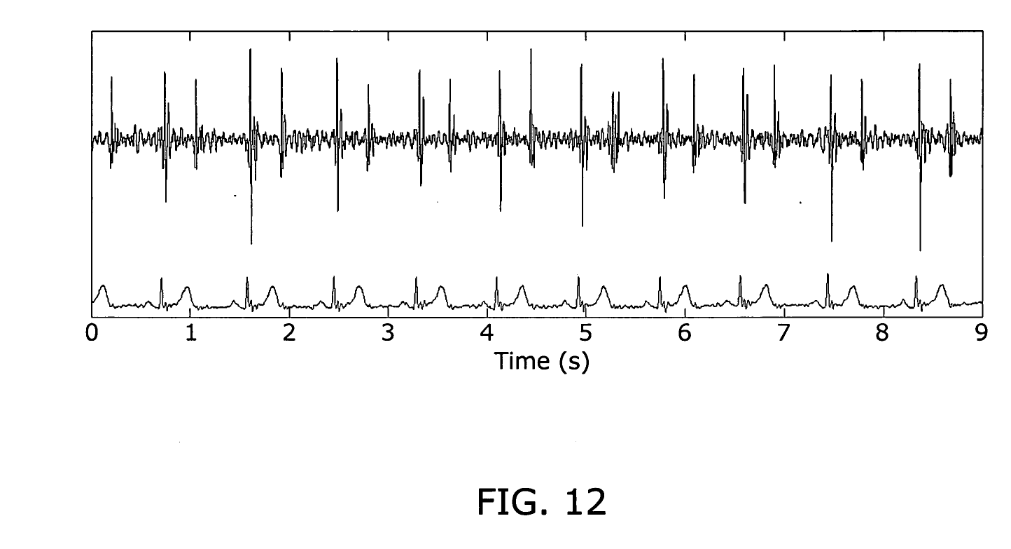

Since the onset of systole, which is marked by the first heart sound (S1), is preceded by the QRS complex, the onset of systole can be detected by detecting the QRS complexes in the EKG. FIG. 12 shows simultaneously recorded acoustic and EKG signals, illustrating the relationship between features of these two signals. (To precisely locate S1 the acoustic signal may be searched to identify a peak immediately after the corresponding QRS complex. However, for many applications this additional accuracy is not required. For example, since the information relevant to the diagnosis of MVP is found in the second half of systole, it is not necessary to distinguish between the QRS complex and S1 since the separation between them is not considered significant.) Any of a variety of methods may be used to detect the QRS complex [16]. In a preferred embodiment of the invention a modified version of the algorithm described by Fraden [17] is employed since it has proven robust in the face of electromyographic noise and powerline interference. The method proceeds as follows:

Let W be a one-dimensional array of sample points of the digitized EKG. An amplitude threshold is calculated as a fraction of the peak value of the EKG signal:

amplitude threshold=0.4 max [W]

The scaling factor of 0.4 corresponds to the optimal value of this parameter determined experimentally in [17].

The raw data is then rectified:

W0(n)=W(n) if W(n)0

W0(n)=−W(n) if W(n)0

Following this, the rectified EKG is passed through a low-level clipper. If W0(n) is greater than or equal to the amplitude threshold:

W1(n)=W(0)n

Otherwise, if W0(n) is less than the amplitude threshold:

W1(n)=amplitude threshold

The first difference is then calculated at each point of the clipped, rectified array as follows:

W2(n)=W1(n+1)−W1(n−1)

Finally, a QRS candidate is declared at every point where W2(n) exceeds the fixed constant threshold:

W2(n)0.33

The value of 0.33 was chosen empirically for the dataset described in section IV based on a trained cardiologist's evaluation of the EKGs. Other suitable values could have been selected, but in general this value will be appropriate for most datasets.

The presence of noise in the EKG signal may give rise to multiple candidates in proximity to actual QRS complexes. These can be removed by adding an extra step to the above method, in which all QRS candidates are discarded except those corresponding to a local peak in the underlying EKG signal. More specifically, if a QRS candidate corresponds to the peak value of the EKG signal over a window having a predetermined width, e.g., 100 ms, centered at the position of the QRS candidate, it is retained. All other QRS candidates are ignored.

The invention provides the following alternative approach: A search is conducted over a window having a predetermined width, e.g., 100 ms, centered at each QRS candidate for the peak value in the EKG signal, and a record of all the peaks found is kept. Nearby QRS complexes map to the same peak, and the positions of the final set of peaks are returned as the locations of the QRS complexes. This approach displays increased tolerance for synchronization errors, using the QRS candidates only as indicators of a nearby QRS complex, which can then be identified by examination of the EKG tracing.

Unlike the case for S1, the EKG does not provide a clear indication of the onset of S2, making its detection considerably more difficult. The invention accordingly provides a new method for locating the second heart sound (S2) within the acoustic signal. As described previously, the T wave precedes S2, but the lag between the T wave and S2 is more variable and longer than the delay between the QRS complex and S1. In other words, S1 occurs at the end of the QRS complex and S2 occurs at the end of the T wave. However, whereas the QRS complex is typically a narrow spike and has an end that is close to its peak in amplitude, there is generally no clear indication of the end of the T wave since it can be arbitrarily wide.

In certain embodiments of the invention this issue is addressed by a method that uses both the acoustic signal and the EKG signal to locate S2. The method, which will now be described, assumes that the positions of the QRS complexes are known. Methods for locating the QRS complex are well known as mentioned above. Denoting the location of the i-th QRS complex in time by qi and the one-dimensional array of points of the digitized EKG by W, in certain embodiments of the invention the method defines the following variables:

beginptᵢ=qᵢ+60 ms

$${endpt}_{i} = {q}_{i} + \frac{2}{3} ( {q}_{i + 1} - {q}_{i} )$$

The candidate T wave corresponding to the i-th QRS complex is then declared to be:

tᵢ=maxpos[W(beginptᵢ:endptᵢ)]

In other words, the T wave corresponding to the i-th QRS complex is declared to be at the position of the peak in the EKG signal between the times beginptᵢ and endptᵢ. It is noted that the values 60 ms and ⅔ in the definitions above are representative only, and other suitable predetermined values can also be used. Thus the method is not limited to the particular parameters in the equations above. In view of the physiological knowledge that the peak in the T wave generally occurs at least 60 ms after the QRS complex and is normally within two thirds of the cardiac cycle immediately following QRS, the parameters selected above may be preferred.

Using this information and denoting the corresponding simultaneously recorded acoustic signal by X, the i-th S2 candidate, sᵢ, is declared to be:

sᵢ=maxpos[X(tᵢ:tᵢ+150 ms)]

S2 is thus declared to be at the position of the peak in the acoustic signal between the candidate T wave and a period of 150 ms following it, where again the use of 150 ms is not intended to be limiting and other predetermined values can be used. However, since it is known that S2 generally lies within approximately 150 ms following the peak of the T wave, values of approximately 150 ms may be preferable.

Rather than locating S2 independently for each systole as described above, in certain preferred embodiments of the invention the median systolic length is calculated and used to approximately predict or estimate the position of S2 following each QRS complex. Using the values of qi and si obtained above, the following parameters are computed:

qslengthᵢ=sᵢ−qᵢ

medlength=median value of qslength

For all i, the position of the i-th S2 using the median systolic length, medsᵢ, is declared to be:

medsᵢ=qᵢ+medlength

In summary, then, the invention provides a variety of methods for locating the second heart sound associated with a beat. One such method comprises: searching over a predetermined time interval following the peak in the T wave for a beat and declaring the position of the second heart sound for the beat to be at the position of the peak in the acoustic signal within the predetermined time interval. A second such method comprises (a) initially searching over a predetermined time interval following the peak in the T wave for each of a plurality of beats and declaring the position of the second heart sound for each beat to be at the position of the peak in the acoustic signal within the predetermined time interval; (b) computing the systolic length for the plurality of beats using the positions of the second heart sounds located in step (a) and the positions of the corresponding QRS complexes or the corresponding first heart sounds; (c) computing the median systolic length for the plurality of beats; and (d) determining the position of the second heart sound for a beat by adding the median systolic length computed in step (c) to the position of the QRS complex or first heart sound corresponding to that beat.

Although the foregoing has described a preferred method of detecting S2, other methods for detecting S2 and/or for detecting systolic be used in the context of the present invention. For example, S2 may be approximately located by using the physiological information that the length of systole is normally approximately 300 ms [50], although when events occurring during the second half of systole are of interest, the variation in the duration of systole between subjects may lead to inaccuracy. Thus methods such as the one described herein, which isolate the position of S2 on a per patient basis, are preferable. It is noted that the inventive methods for identifying S2 are useful in a variety of contexts and are not limited to use in the present invention.

The preceding description has focused on the inventive methods for segmenting the acoustic signal into systolic regions. It will be appreciated that similar approaches may be used to segment the acoustic signal into diastolic regions. While segmentation into systolic or diastolic regions may often be sufficient, it may be desirable to further segment the acoustic signal into subregions. This may be done in a variety of ways including using additional features of the EKG, selecting time intervals on either side of the QRS complex, S1, or S2, etc.

(2) Noisy Beat Rejection and Length-Biased Beat Admission

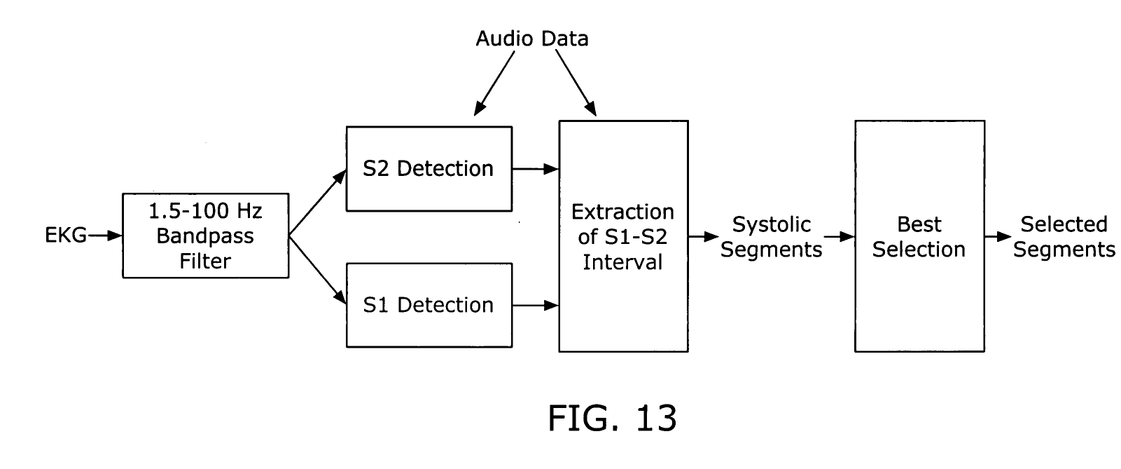

The methods described above allow isolation of a region of interest such as the systolic portion of the acoustic signal for further analysis as shown in FIG. 13, which depicts a block diagram of the system to extract systolic segments with beat selection. In preferred embodiments of the invention a screening step is performed prior to the analysis stage that determines whether or not any particular beat should be examined further. Although the description herein assumes screening after segmentation, the beats could also be screened prior to segmentation.

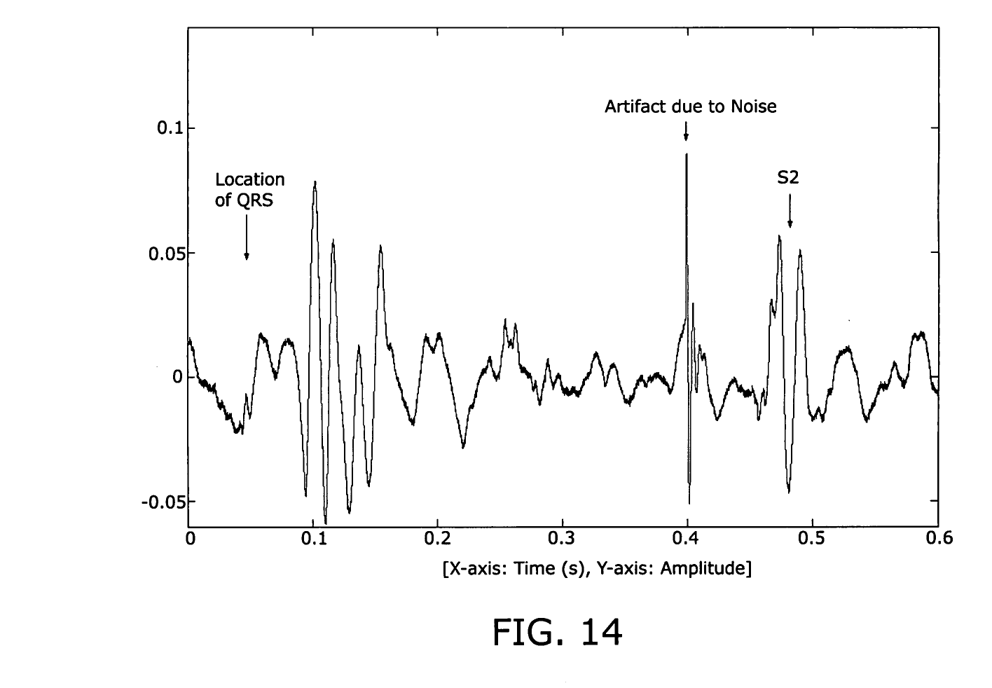

Any suitable method for detection of noise in a beat can be used. According to one approach, the invention uses the information that cardiac events during the middle of systole typically do not have significantly greater energy than the first and second heart sounds. This represents another instance of the use of physiologically and/or mechanistically relevant information in the inventive methods. According to certain embodiments of the invention beats with peak amplitudes during the middle half of systole that are greater than the amplitude of both heart sounds are declared to be noisy. (Other criteria could be used. For example, beats could be declared noisy if their peak amplitudes during a time period of interest exceed a predetermined value or a predetermined fraction of the amplitude of one or both of the heart sounds.) The increase in energy is attributed to the presence of artifacts (noise) and the beats are discarded. FIG. 14 provides an example of a beat labeled as being noisy by the invention.

According to one approach, starting with the positions of the QRS complex and S2 corresponding to the i-th beat, to determine whether that beat is noisy, the length of the systolic portion of the beat is first calculated as follows:

systoliclength=sᵢ−qᵢ

Using this value and letting X be a one-dimensional array corresponding to the acoustic signal, the peak amplitude of the beat during the middle half of systole is defined as:

$${maxpeakamp}_{i} = max [ X ( {q}_{i} + \frac{systoliclength}{4} : {S}_{i} - \frac{systoliclegth}{4} ) ]$$

The amplitude of the first heart sound (which generally lies within a period of approximately 100 ms after the QRS complex) is then found by:

s1ampᵢ=max[X(qᵢ:qᵢ+100 ms)]

Since sᵢ is chosen to approximately correspond to the peak of S2, the amplitude of the second heart sound can be determined by conducting a localized search over a window of 50 ms centered at sᵢ:

s2ampᵢ=max[X(sᵢ−25 ms:sᵢ+25 ms)]

If maxpeakampᵢ is more than the value of both s1ampᵢ and s2ampᵢ, the beat is discarded. The value of 50 ms was selected based on the observation that the maximum shift of S2, for any beat, from the predicted position of S2 determined as described above is typically less than 50 ms. However, it is noted that the values presented herein are not intended to be limiting but present only one of a number of possible choices.

In addition to removing noisy beats, the inventive methods also select beats based on a variety of other criteria. For example, it may be preferable to exclude beats whose duration varies greatly from that of the mean or median beat length, or it may be desirable to include beats selectively based on their length. The invention encompasses the recognition that beats of different lengths may have different information contents, and that the information content may vary depending upon the disease or clinical condition to be evaluated. For example, in the case of MVP, beats having systolic segments that are preceded by a long diastole may be preferred to those following shorter diastolic periods. A longer diastole allows the ventricles more time to fill with blood, leading to an increase in the volume of blood passing through the heart, which in turn produces a more audible murmur in the presence of MVP [ 18, 19]. Here again physiologically or mechanistically relevant information is used in the methods of the invention.

Selection of beats based on their length and/or the length of a region of interest within the beat is referred to herein as length-biased beat selection. Any of a variety of methods for selecting beats based on length may be used. For example, beats longer or shorter than predetermined thresholds may be included. The thresholds may be established based on metrics such as mean or median beat length, standard deviation in beat length, etc. Without intending to be limiting, one inventive method to achieve length-biased beat selection starts by calculating the median length for the R-R intervals. (The R-R interval for any beat is defined as the distance from the R wave of the previous beat to the R wave of the current one.) Letting W(i) be the length of the R-R interval associated with the i-th beat, define:

W_{median}=median [W(i)]

W_{deviation}=standard dev [W(i)]

Following this, first upper and lower thresholds Wᵤₚₚₑᵣ and W_{lower} are set that control which beats are selected. The thresholds are selected based on the median and standard deviation of the beat length. The numerical values presented in the equations below are representative of values suitable for use in the method:

Wᵤₚₚₑᵣ=W_{median}+0.3 W_{deviation}

W_{lower}=W_{median}−0.3 W_{deviation}

Beats within the range between W_{lower} and Wᵤₚₚₑᵣ, optionally including beats on the limits of the range, are further examined as discussed below. In general, it is preferable to analyze several beats. For example, and without intending to be limiting, in one embodiment of the invention 20 beats are analyzed for every second of recorded acoustic signal. Since the initial range between W_{lower} and Wᵤₚₚₑᵣ frequently contains fewer beats, the invention defines second upper and lower thresholds for widening the range of admission:

Wᵤₚₚₑᵣ′=Wᵤₚₚₑᵣ+0.05 W_{deviation}

W_{lower}′=W_{lower}−0.025 W_{deviation}

This step introduces bias in favor of longer beats. In particular, the range of admission is widened by 0.05 times the standard deviation upwards but only by half that factor in the lower direction. By using a larger numerical value in the computation of W_{lower}′, selection would be biased in favor of shorter beats. It is noted that the numerical values in the equations for the first and second upper and lower thresholds may vary, and a number of different predetermined values can be used. Length bias will be introduced at the step of calculating either or both the first and second upper thresholds if the predetermined values used in the computation of the upper and lower thresholds are different.

Although use of an upper threshold is not necessary in order to select longer beats, it may be preferable to reject the longest beats in the signal if they differ significantly from the R-R interval as they may be outliers or represent segmentation errors. In general, beats whose length deviates greatly from the mean or median length may be rejected.

(3) Additional Features of the Beat Selection Component

Although the beat selection component described above operates by either admitting or rejecting a beat for analysis, a variety of other features may be incorporated. For example, the beat selection component may weight beats rather than simply admitting them. The weighting may give increased diagnostic significance to beats having certain features, e.g., length, absence of noise. In addition, various physiological criteria other than beat length may be used to decide whether to admit particular beats and/or what weight to assign to them. Information such as the position of the patient when the acoustic signal was acquired, the timing of the beat in relation to respiration, etc. may be used to select and/or weight beats. For example, it is known in the art that the degree of “split” in the second heart sounds, caused by the different time at which the aortic and pulmonic valves close, varies with respiration. This fact may be used to differentiate between individuals who have a split S2 and those that have MVP. It is noted that detection of breath sounds may provide useful diagnostic information independent of its utility in beat selection. The beat selection component may also determine which beats are appropriate for examination in the context of significant arrhythmia. It will be appreciated that in such cases many beats may lack significant diagnostic information or may contain misleading diagnostic information.

In addition to rejecting beats classified as too noisy, in certain embodiments of the invention techniques such as adaptive signal processing [55] may be used to remove noise from the signal.

C. Time-Frequency Analysis

Following selection of beats, the system performs a time-frequency analysis, which includes a time-frequency decomposition of the selected beats. In general, the time-frequency decomposition of a signal provides information regarding the distribution of energy with respect to time in a plurality of frequency bands.

(1) Filter Bank

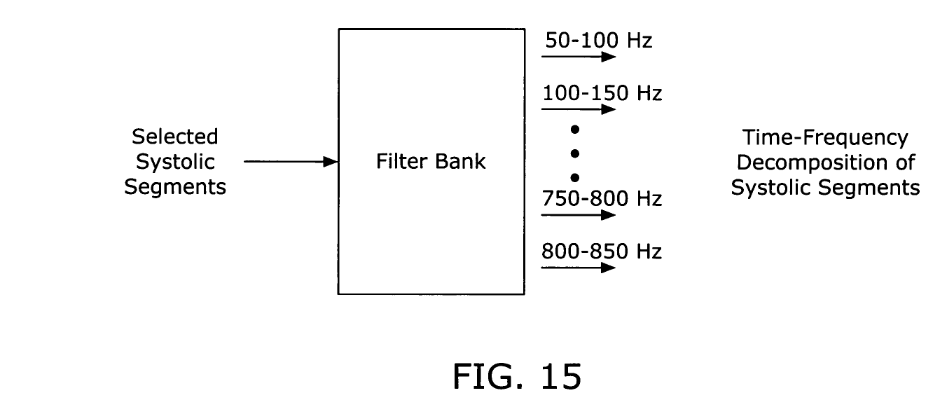

According to certain embodiments of the invention, in order to achieve a time-frequency decomposition of acoustic signals a filter bank that separates the acoustic signal into its constituent frequency bands is used. The filter bank comprises a series of sharp frequency filters to substantially reduce or eliminate overlap between bands. For example, in one embodiment of the invention 16 bands are used, each spanning a 50 Hz interval from 50 to 850 Hz. Each filter corresponds to a finite impulse response approximation to the infinite impulse response of an ideal filter [20] in which the length of the Hamming windows is 50001. This leads to filters with a peak approximation error of −53 dB and transition bands with a width of approximately 3.5 Hz. FIG. 15 shows the block diagram for this filter bank.

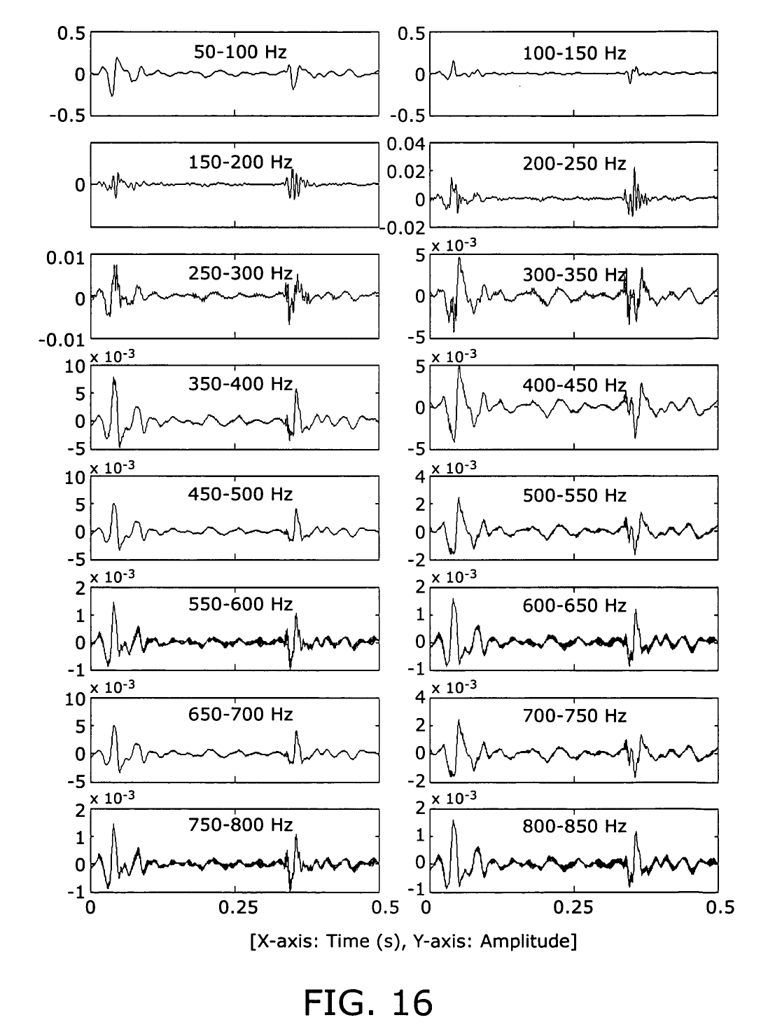

It will be appreciated that many different filter types may be used in the filter bank, and the number of filters can vary. The filters may all have the same widths or they may have different widths. In preferred embodiments of the invention the width of the transition band for each of the filters should be significantly lower than the total width of each frequency band. In other words, the transition from passband to stopband should be sufficiently sharp so as to prevent the energy content of any frequency band from containing a significant contribution from adjacent bands. This is particularly important when the amount of energy in two adjacent bands is very different. Since the amplitude of low frequency in the acoustic signal is generally several orders of magnitude greater than the amplitude of high frequency energy (FIG. 16) a transition band that is too wide (e.g., one that does not significantly attenuate the low frequency content of the signal received from neighboring bands) would lead to low frequency energy swamping the energy at higher frequencies, potentially concealing trends at higher frequencies and reducing the visibility of high frequency components that may be indicative of clinical conditions such as heart murmurs (e.g., murmurs associated with MVP). Thus according to certain embodiments of the invention the energy content of the signal passed by each frequency filter is substantially free of energy contributed by signal from neighboring frequency bands. In various embodiments of the invention by “substantially free” is meant that at least 70%, at least 80%, at least 90%, at least 95%, or at least 99% of the energy content of the signal passed by each frequency filter is contributed by signal from that filter's frequency band. According to certain embodiments of the invention the transition bands of the frequency filters have widths less than approximately 5%, less than approximately 10%, less than approximately 20%, or less than approximately 30% of the widths of their passbands and/or the passbands of adjacent frequency filters.

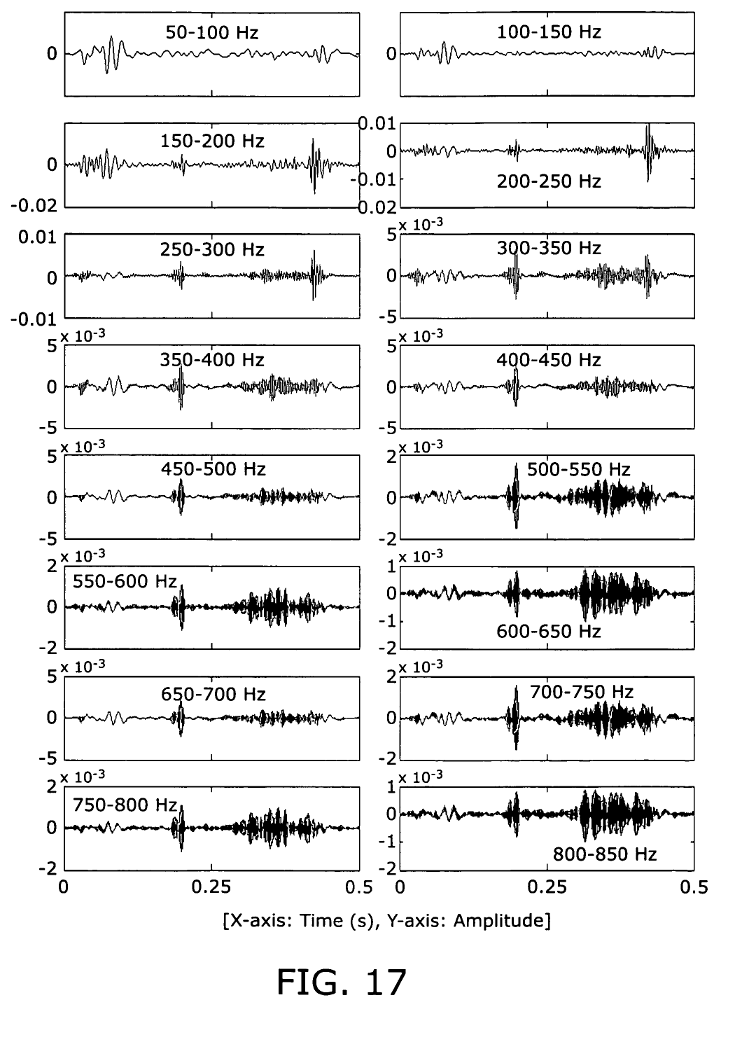

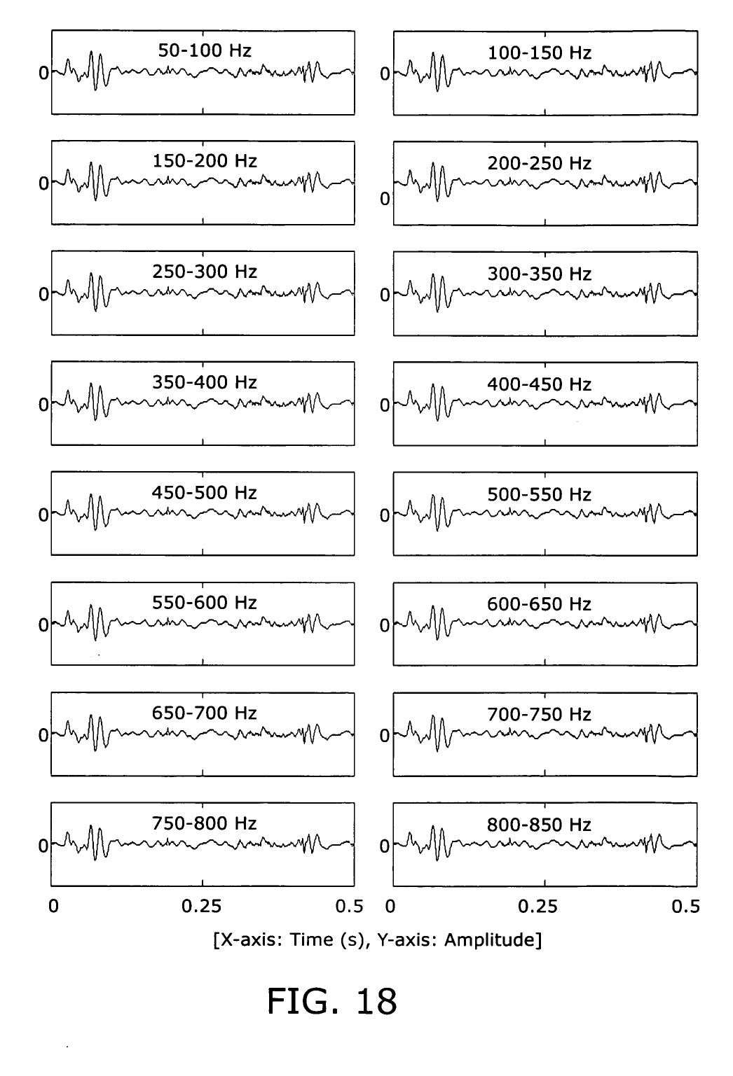

FIGS. 17 and 18 illustrate the effect of using filters that fail to meet the criteria discussed above for a patient suffering from moderate MVP with late systolic regurgitation. FIG. 17 displays the time-frequency decomposition achieved for a filter bank using Hamming windows of length 50001 (corresponding to a transition band of width 3.5 Hz for each filter). The presence of high frequency energy just prior to S2 can be readily discerned from the plots, thus it can be recognized that the patient suffers from MVP. FIG. 18 shows the time-frequency decomposition for a filter bank employing Hamming windows of length 1001 (transition band width of 176.2 Hz). It can be seen that at lower frequencies there is little difference between the two figures. However, in FIG. 18, energy at lower frequencies suppresses information in the higher frequency bands, making it difficult to identify the presence of signal indicative of MVP. The suitability of different filters for use in the present invention may readily be tested by evaluating the performance of the system using a set of acoustic signals from patients known to suffer from the clinical condition(s) whose presence or severity the system is designed to evaluate as described later herein.

Although use of a filter bank is preferred in certain embodiments of the invention, other methods for performing time-frequency decomposition and/or analysis may also be used, e.g., Short Time Fourier Transform, wavelets, etc.

(2) Band Aggregation

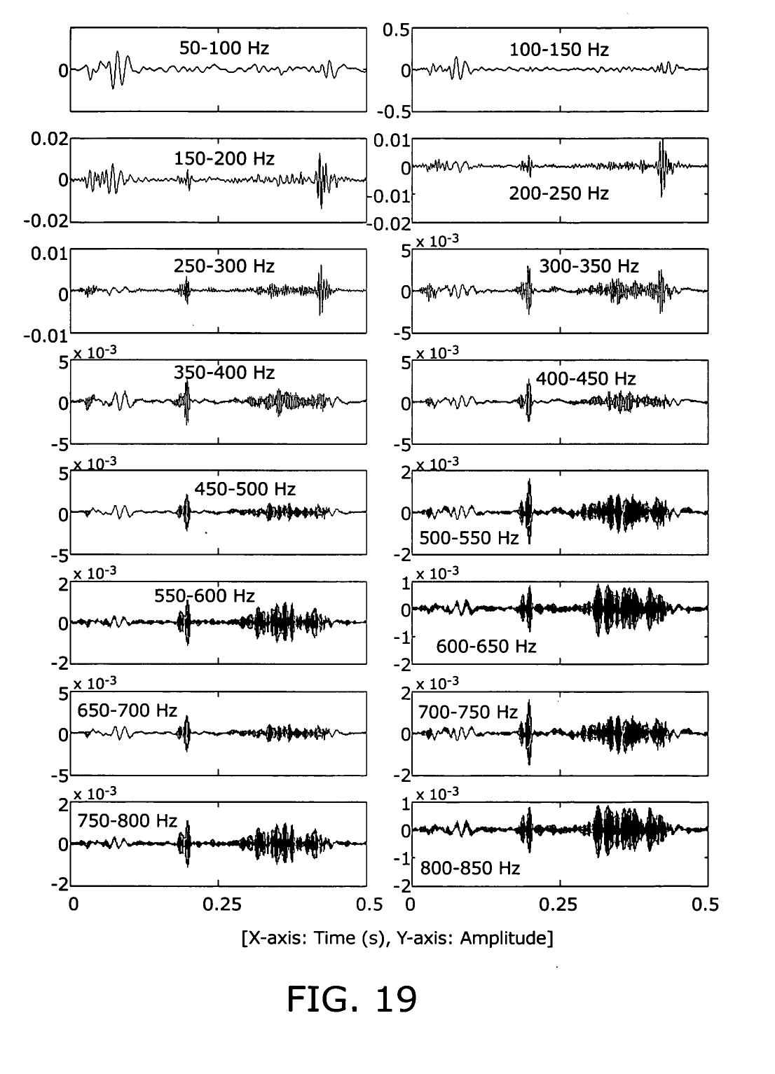

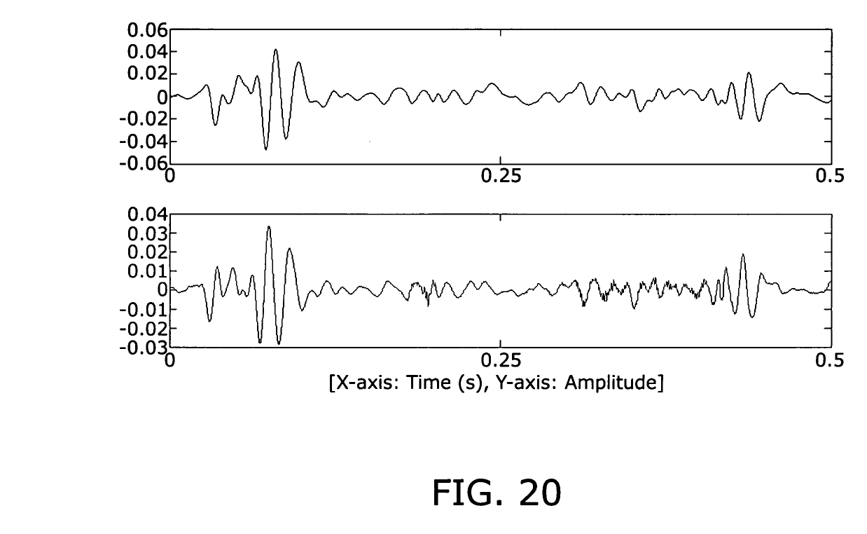

The filter bank described above divides the signal into a plurality of frequency bands, each spanning a portion of the frequency region in which acoustic signals of interest in the diagnosis of conditions of the cardiovascular system are found. In those embodiments of the invention in which band aggregation (see below) is employed, these bands may be referred to as initial frequency bands. For example, the filter bank shown in FIG. 15 divides the signal into 16 bands, each spanning 50 Hz. A smaller number of wider bands or a larger number of bands each spanning a smaller interval could also be used. The particular choice made reflects a tradeoff between various factors such as performance and computational requirements as discussed below. It may be preferable to divide the acoustic signal into a relatively large number of bands such as 16, as opposed for example, to just a low frequency band and a high frequency band because heart murmurs and other acoustic signals emanating from the CV system that may be indicative of the presence or severity of a clinical condition may differ in the range of frequencies over which they lead to an increase in energy. If these ranges are sufficiently narrow, aggregating all the frequency bands together into a single band obscures or eliminates useful diagnostic information. This effect is illustrated in FIGS. 19 and 20, which once again display the time-frequency decomposition corresponding to the patient shown in FIGS. 17 and 18. Although the presence of high frequency energy in systole prior to S2 is clearly visible in FIG. 19, aggregating the higher frequency bands obscures it in FIG. 20.

Maintaining a sufficiently fine granularity in frequency offers the possibility of providing information beyond a determination of whether a particular disease or condition exists, e.g., qualitative or quantitative information regarding the severity or extent of the condition. For example, specifically identifying the frequencies at which MVP leads to an increase in energy and observing the extent of that increase provides information regarding the size of the opening between the leaflets of the mitral valve and the corresponding volume of regurgitation.

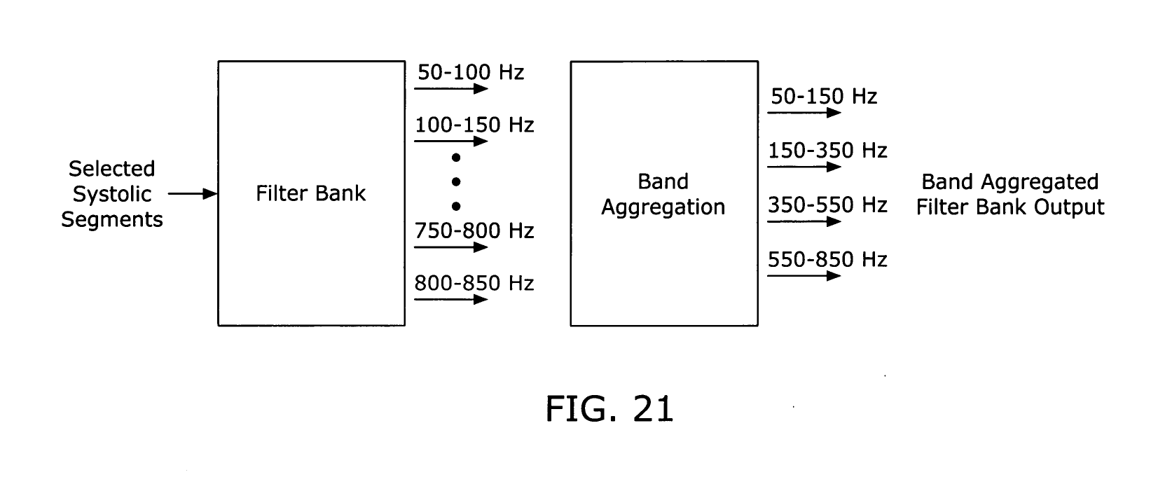

In certain embodiments of the invention it may be preferred to aggregate certain of the initial frequency bands. This approach may enhance performance in the presence of noise, e.g., high frequency noise. Empirically, such noise appears to be localized in a narrow range of frequencies and appears to corrupt only one or at most two of the bands output by the filter bank. In such a situation, band aggregation (merging multiple bands together) enhances performance. According to one approach, limited band aggregation is used. For example, in the embodiment described above in which a 16-filter bank is employed, the following four composite bands FIG. 21 are created:

50-150 Hz

150-350 Hz

350-550 Hz

550-850 Hz

It will be appreciated that the composite bands may have the same or different widths and may merge the same or different numbers of adjacent bands. For example, in the embodiment described above the bands differ in width because the signal to noise ratio was generally worse at frequencies above 600 Hz. Since the energy in the low frequency bands is generally several orders of magnitude greater than the energy in bands at higher frequencies, in preferred embodiments of the invention the process of aggregation performs normalization prior to combining different bands together. Failure to do so would result in the lower frequencies dominating the higher ones when composite bands are formed. Dividing the acoustic signal into a set of initial frequency bands and then aggregating those bands to form a smaller number composite bands following normalization may thus be preferred to the alternate approach of using a filter bank that directly outputs the smaller number of bands. Since none of the murmurs in the dataset described below displayed a localized increase in energy that necessitated finer granularity than 50 Hz, little would have been gained by dividing the signal into more than 16 bands and aggregating these together, and doing so would have increased computational costs.

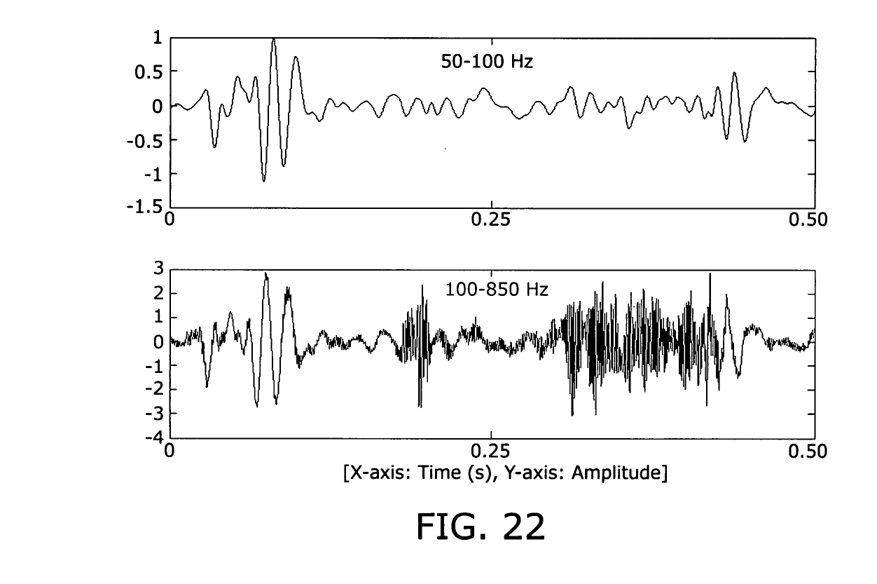

One approach to normalization (referred to herein as the fair aggregation approach) involves scaling bands by the reciprocal of their maximum value prior to combination. FIG. 22 shows the effect of this approach when used to aggregate all frequency bands above 100 Hz into a single band for the patient shown in FIG. 20. This method achieves better results (less loss of diagnostic information) than the non-normalized aggregation illustrated in FIG. 20, in which the signal in the bands that are to be aggregated are simply added without scaling.

The operation of this fair aggregation approach to create a composite band of 150-350 Hz will now be described. Defining X₁₅₀(n), X₂₀₀(n), X₂₅₀(n) and X₃₀₀(n) as the outputs of the filter bank corresponding to the 150-200 Hz, 200-250 Hz, 250-300 Hz and 300-350 Hz bands, the composite band X₁₅₀₋₃₅₀ is given by:

$${X}_{150 - 350} = \frac{{X}_{150}}{max [ {X}_{150} ]} + \frac{{X}_{200}}{max [ {X}_{200} ]} + \frac{{X}_{250}}{max [ {X}_{250} ]} + \frac{{X}_{300}}{max [ {X}_{300} ]}$$

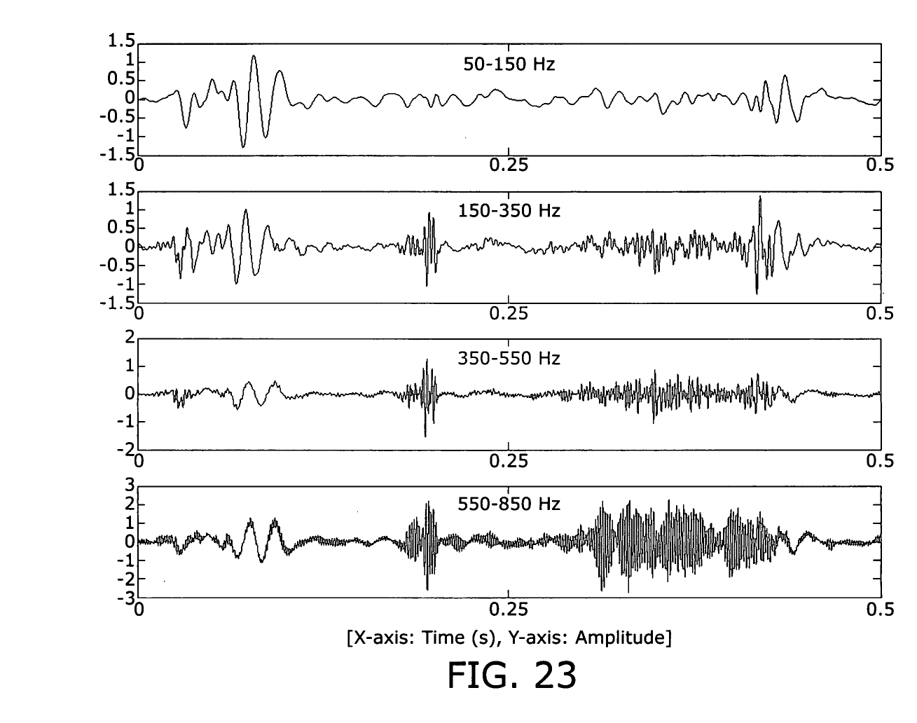

FIG. 23 displays the output of this band aggregation approach for the patient discussed above suffering from moderate MVP with late systolic regurgitation. In order to reduce destructive interference (i.e., positive and negative values from different bands canceling each other), the absolute value at every time instant for each band output by the filter bank (i.e., the time-envelope characterization for these bands) can be calculated and passed to the ensuing stages. Since the system focuses on the energy content of the signal, which is reflected by its amplitude, it does not, in general, matter whether the signal is positive or negative at this stage.

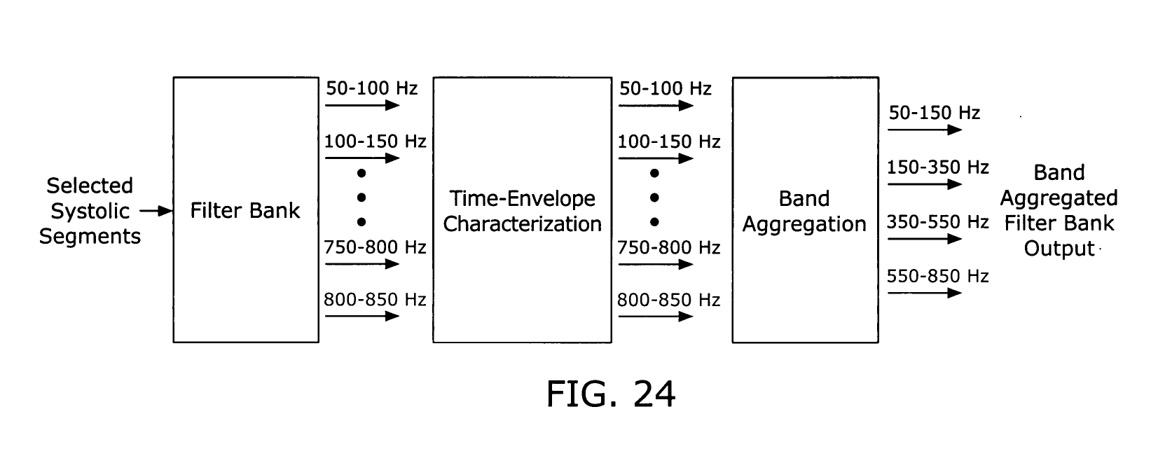

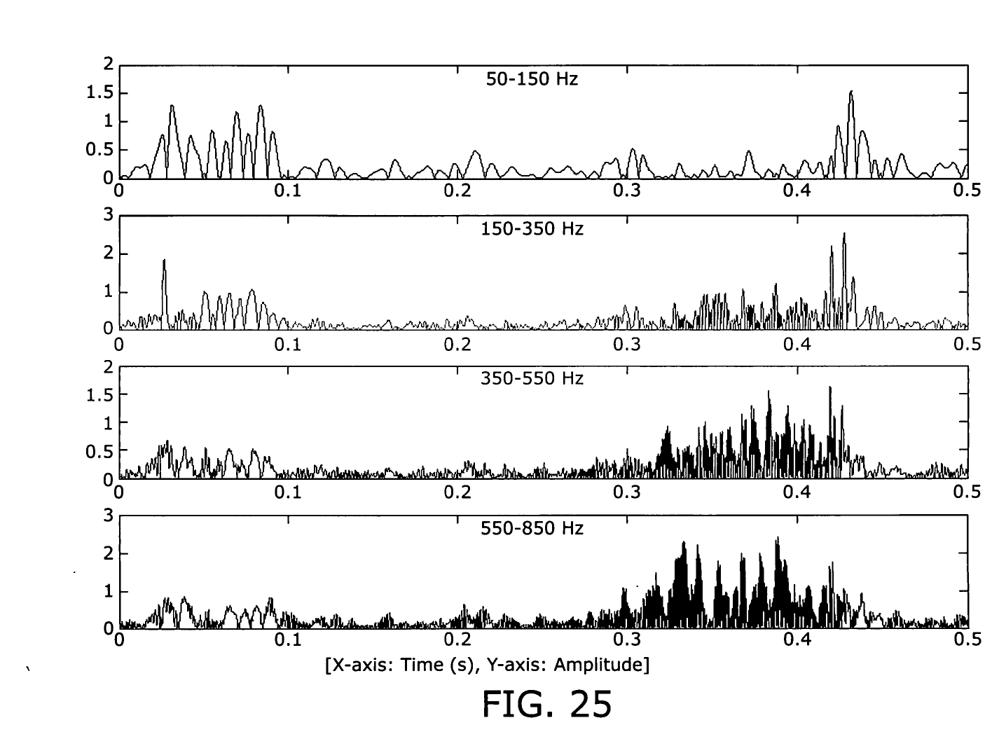

A block diagram representation of the time-frequency decomposition components of the system is presented in FIG. 24. FIG. 25 shows the output of the limited band aggregation band approach with time-envelope characterization described above for the same acoustic signal displayed in FIG. 23.

D. Beat Aggregation

The time frequency decomposition discussed above provides the frequency components for each selected beat. In order to observe the characteristic trends persisting among multiple beats (e.g., the majority of beats), the invention also provides a method for merging information from multiple beats or regions of interest thereof to create a single representative beat for the subject. This beat will be referred to herein as a prototypical beat. According to one approach, the method assimilates information from the selected beats to generate the time-frequency decomposition of a hypothetical “typical” beat for the subject.

The method for beat aggregation begins with the time-frequency decomposition of selected beats as described above. Either some or all of the selected beats can be used. The time-frequency decomposition divides each beat into its band-aggregated components at different frequencies. These components are time-envelope characterized, i.e., the absolute value at every time instant for the component signals is calculated. For purposes of description, it will be assumed that composite bands of 50-150 Hz, 150-350 Hz, 350-550 Hz, and 550-850 Hz have been created. The beats are then lined up in time, and a subset of amplitudes at any time instant for each of the bands is determined. For example, a median set of amplitudes at any time instant may be computed. The median set may consist of the median X amplitudes at any time instant, where X can be any number less than the total number of beats. For purposes of description herein, it will be assumed that X=4.

One approach to addressing the possibility that the beats may be of different lengths is to assume that all beats have the same length, i.e., to truncate longer beats. A second approach is to resize the beats such that they are all of the same length. To do this, the same methods as the ones employed to slow down heart sounds without loss of frequency content (described in Section IV) may be used. For example, shorter beats may be slowed down until their lengths are equal to the length of the longest beat. This would lead to all beats having the same length, without the loss of important frequency information for any beat.

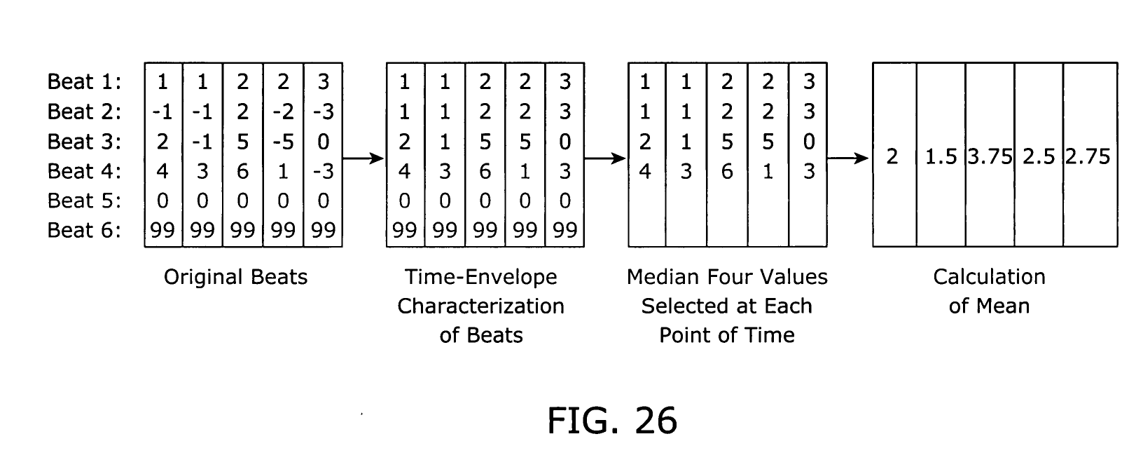

The overall process of calculating the prototypical beat is depicted in FIG. 26. Letting X_{i,j} be a one-dimensional array corresponding to the j-th frequency band of the i-th beat, the step of prototypical beat calculation can be represented as finding the median four elements along every column of the array:

$$( \begin{matrix}{X}_{1 , j} \\ {X}_{2 , j} \\ ⋯ \\ {X}_{n - 1 , j} \\ {X}_{n , j}\end{matrix} )$$

for all possible values of j, i.e., 50-150 Hz, 150-350 Hz, 350-550 Hz, and 550-850 Hz. The mean of these median amplitudes is then calculated for each range of frequencies output by the filter bank. This is illustrated on the right in FIG. 26. Representing Y_{1,j}, Y_{2,j}, Y_{3,j}, and Y_{4,j} as one-dimensional arrays that each contain one of the median four elements at every time instant for the j-th band, this step corresponds to calculating the mean of the array:

$$( \begin{matrix}{Y}_{1 , j} \\ {Y}_{2 , j} \\ {Y}_{3 , j} \\ {Y}_{4 , j}\end{matrix} )$$

for all possible values of j.

The result is a time-frequency decomposition of the prototypical beat. If Zⱼ is the one-dimensional array containing the means of the median four values calculated at every time instant for the j-th band, the prototypical beat has a time-frequency decomposition given by:

50-150 Hz: Z1

150-350 Hz: Z2

350-550 Hz: Z3

550-850 Hz: Z4

Pooling multiple beats allows the derivation of a representation of the sound actually generated by the heart while discarding random or systematic noise. Since only a median set of amplitudes at each time-band pair is examined, artifacts leading o increased energy in the signal are treated as outliers and are removed except in the circumstance where these artifacts occur at precisely the same instant in the cardiac cycle for at least 50% of the beats, which is unlikely to occur.

Taking the mean of the median set of beats adds further robustness to noise. According to another approach, the mean of the overall signal rather than the mean of the medians is used to calculate the prototypical beat. However, this approach appears more susceptible to artifacts. Median filters are well suited to address noise that falls into the category of impulsive, salt-and-pepper noise, such as that frequently observed in acoustic signals emanating from the CV system [21, 22, 23, 24]. The components of the time-frequency decomposition of the prototypical beat may be added together to generate a complete prototypical beat. Prior to such addition the bands may be normalized, e.g., by dividing each band by its maximum amplitude.

Although the construction of the prototypical beat loses information regarding variation between beats, it does not, in general, result in a significant loss of relevant information. Patients suffering from a wide variety of disorders and conditions of the cardiovascular system, including mitral valve prolapse, show evidence of the disorder on the majority of recorded beats. In such a case calculation of the median beats leads to a subset of beats that all possess the signature features of the condition. Conversely, though noise might cause normal patients to have one or more beats that appear to contain the signature features of the condition (e.g., energy at higher frequencies in the time interval prior to systole in the case of MVP), it is less likely that the median set of beats would all suffer from this effect.

E. Decision Mechanism

The processes of beat selection and time-frequency analysis provide information regarding how energy is distributed over different frequency ranges for beats belonging to the subject. The beat aggregation mechanism reveals persisting trends in the recorded signals. This section describes methods for processing this information in order to reach a clinically relevant conclusion or recommendation. In general, during such processing the decision mechanism computes one or more metrics or indicators, e.g., time metrics, amplitude metrics, or both, that characterize the distribution of energy at one or more points in the cardiac cycle in at least one of the frequency bands.

By “metric” or “indicator” is meant a measurement or qualitative indication of a particular characteristic of the energy distribution, generally a characteristic that is useful in distinguishing between subjects who do or do not have a disease or condition of the cardiovascular system, or a characteristic that reflects the severity or extent of such a condition. The metric may reflect the distribution of energy over time in different frequency bands or the amplitude of the energy. For example, it may reflect the time at which the energy exceeds a particular threshold value, the peak energy value reached, the time at which the peak energy value is reached, duration of various components of the signal, etc. Other metrics include the existence and magnitude of a crescendo or decrescendo in a region, the existence or magnitude of harmonic energy (i.e., existence of periodicity in the signal) in particular frequency ranges, etc. While not wishing to be bound by any theory, it is noted that in the case of certain conditions the signal from benign murmurs in the high frequency bands is more likely to be periodic.

It will be appreciated that any of a large number of different metrics may be employed, and the particular choices will vary depending on the condition to be evaluated. In general, the metric may be used to classify individual beats or a prototypical beat as indicative of the presence (or absence) of a disease or condition of the cardiovascular system. For example, if the value of the metric for a particular beat exceeds or falls below a predetermined value characteristic of normal subjects, the beat may be classified as indicative of the presence of a condition or disorder. The metric may also be used to assess the severity or extent of the condition on a per-beat basis or based on a prototypical beat.

(1) Band-Specific Thresholding

According to certain embodiments of the invention a method referred to as band-specific thresholding is employed to compute a metric that may be used to classify beats. While this approach and many of the specific elements of the method are broadly applicable to a wide variety of conditions and diseases of the cardiovascular system characterized by abnormal acoustic signals, the method will be described with reference to an embodiment of the invention that diagnoses mitral valve prolapse.

Since MVP is characterized by increased energy content at higher frequencies during the last half of systole, the method focuses on locating peaks in energy at higher frequencies for every selected beat. The position of maximum signal amplitude is determined separately for each range of frequencies output in the time-frequency analysis. For MVP, this search is limited to a region from mid-systole to slightly after S2 since all diagnostic information is expected to be present there. In particular, if X_{i,j} is a one-dimensional array of acoustic data corresponding to the j-th frequency band of the i-th selected beat (i.e., an array containing the amplitude of the acoustic signal for the systolic segment between the i-th S1 and S2 with only the components in the j-th band included), define the variable peakpos_{i,j}, the position of maximum amplitude for the j-th frequency band of the i-th beat to be:

$${peakpos}_{i , j} = maxpos [ {X}_{i , j} ( \frac{length ( {X}_{i , j} )}{2} : length ( {X}_{i , j} ) ) ]$$

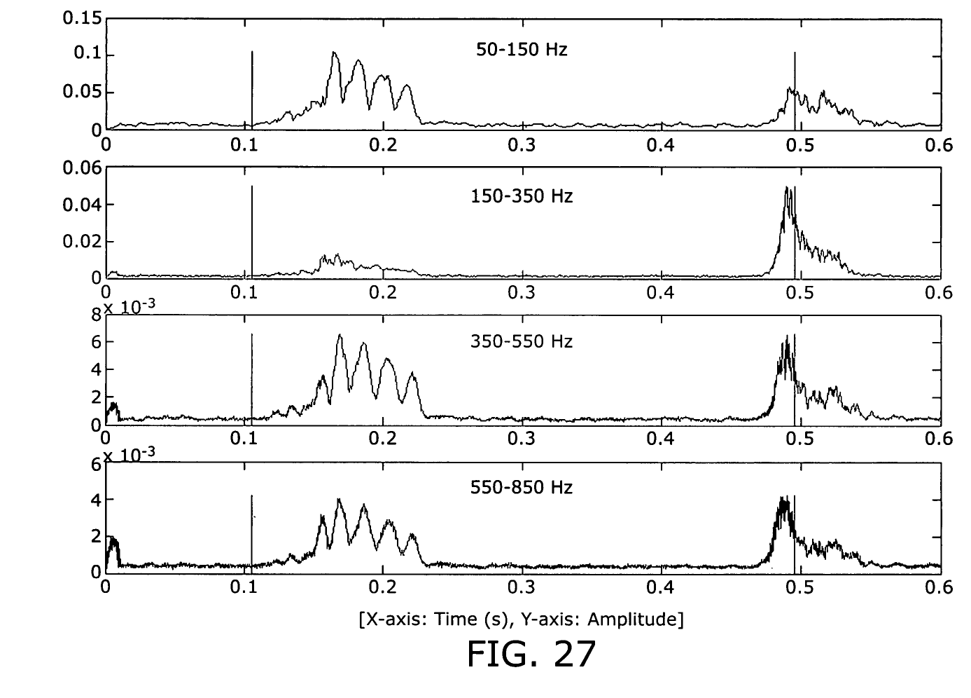

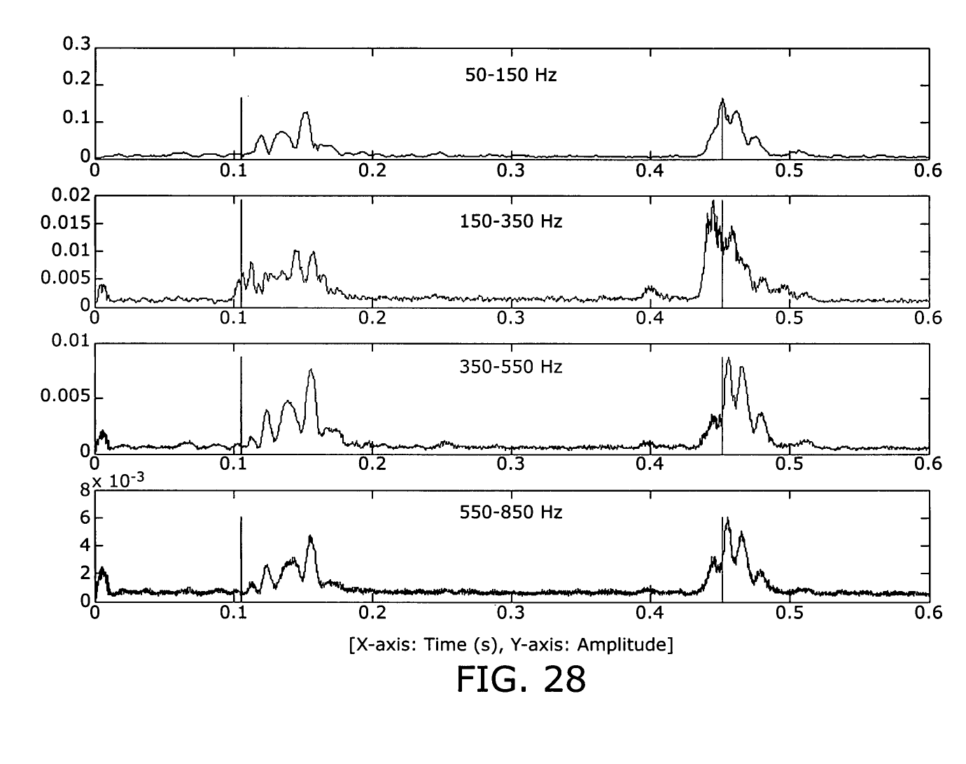

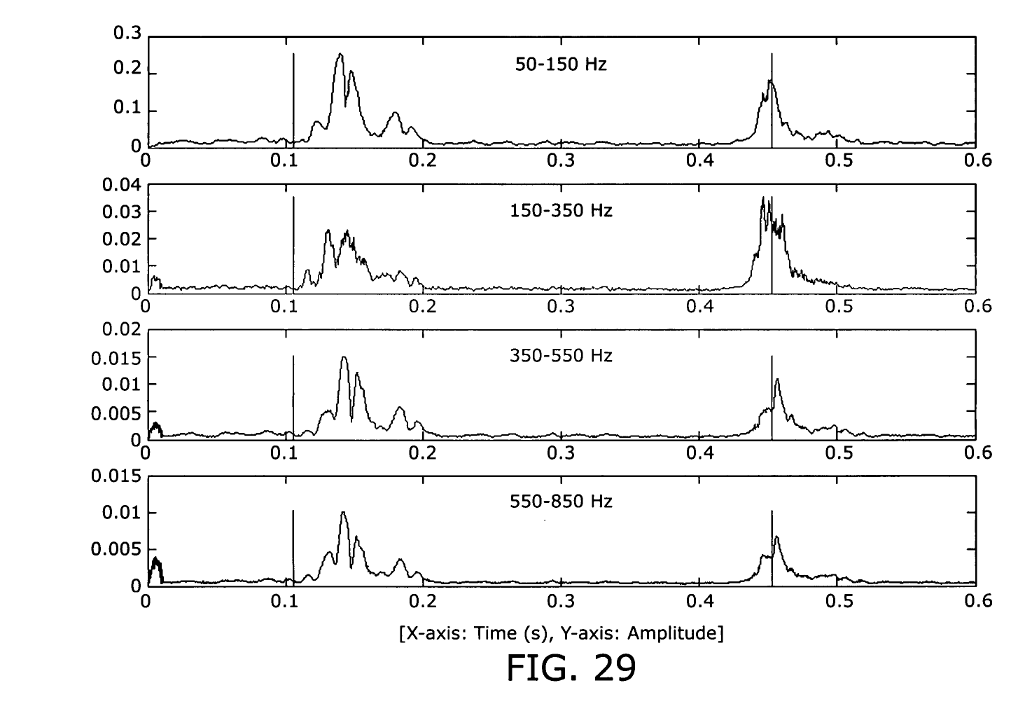

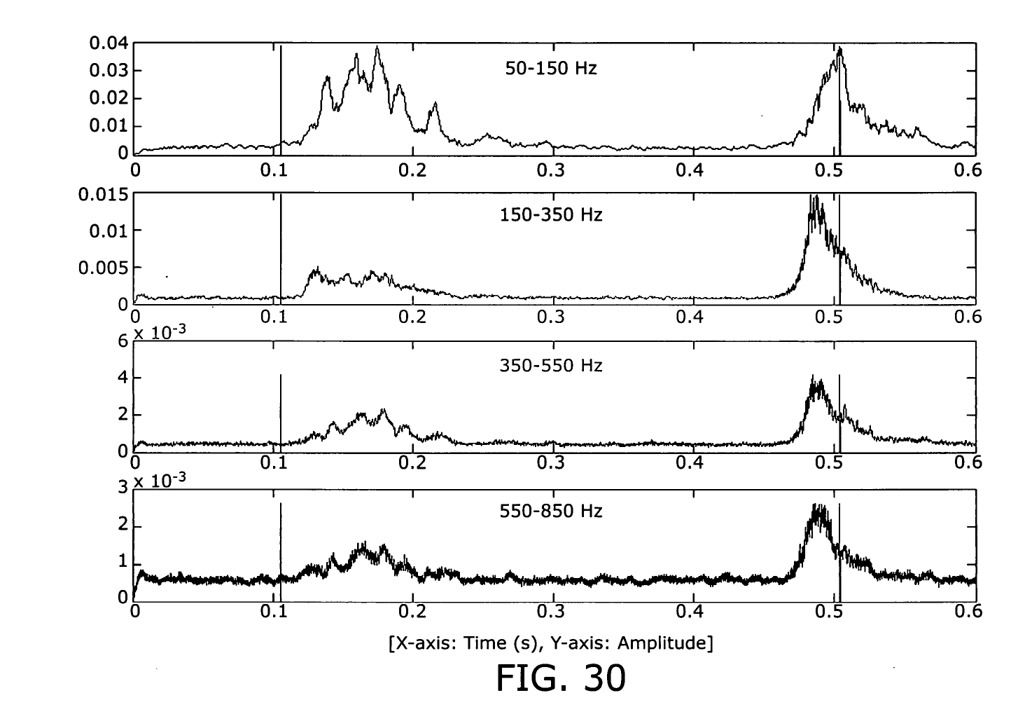

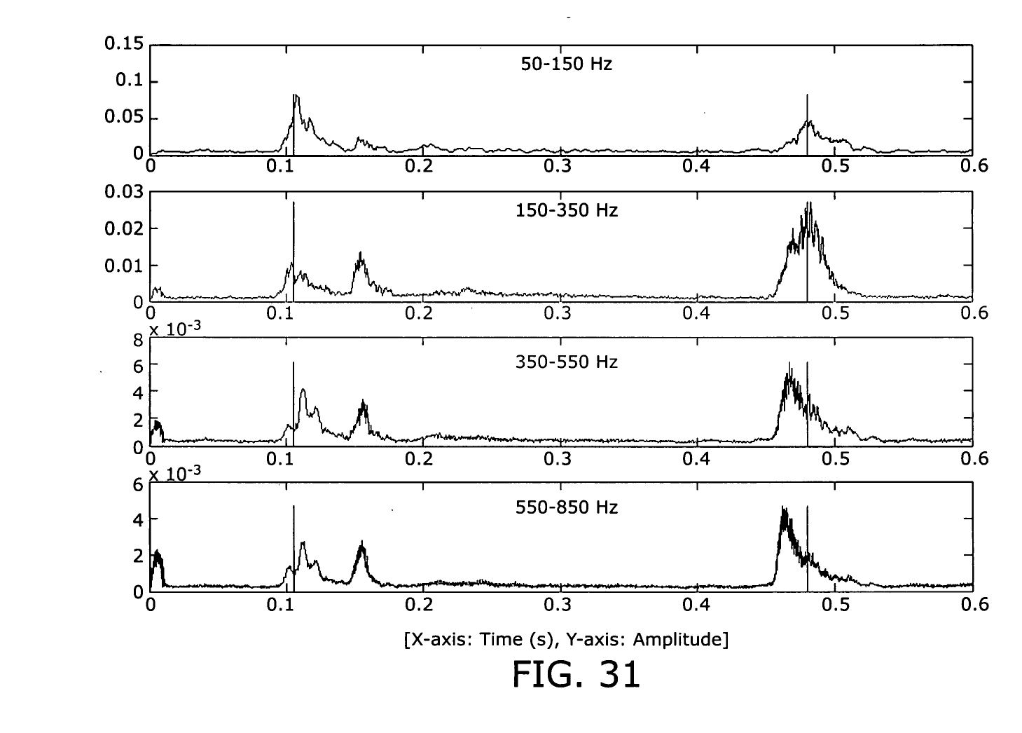

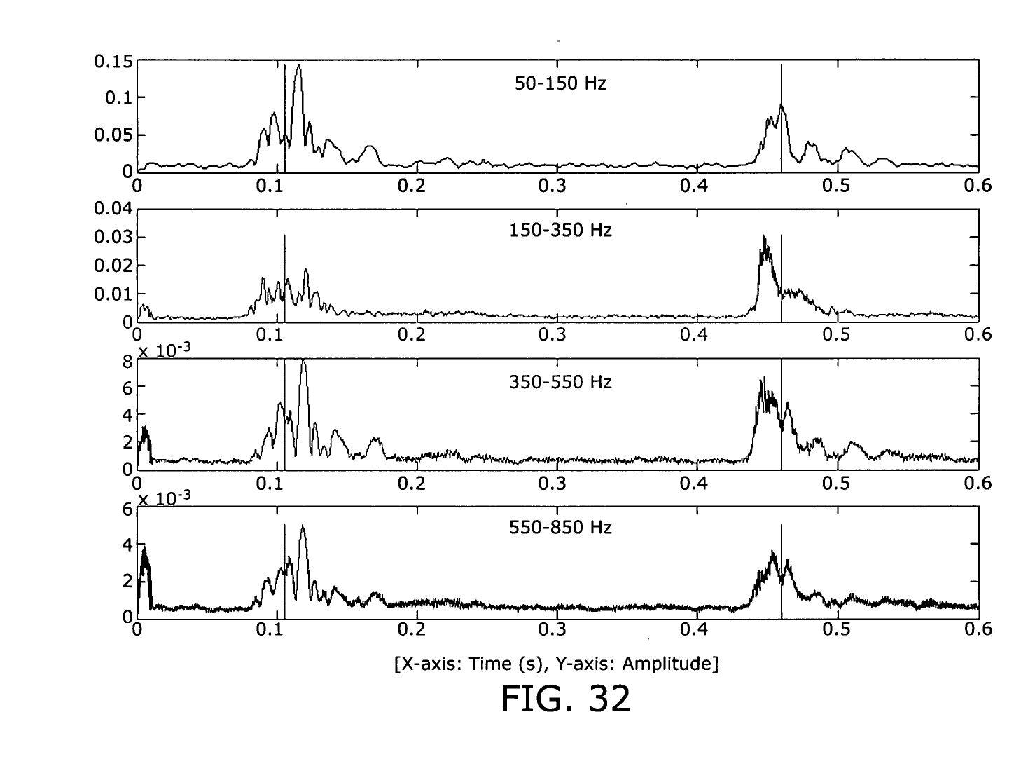

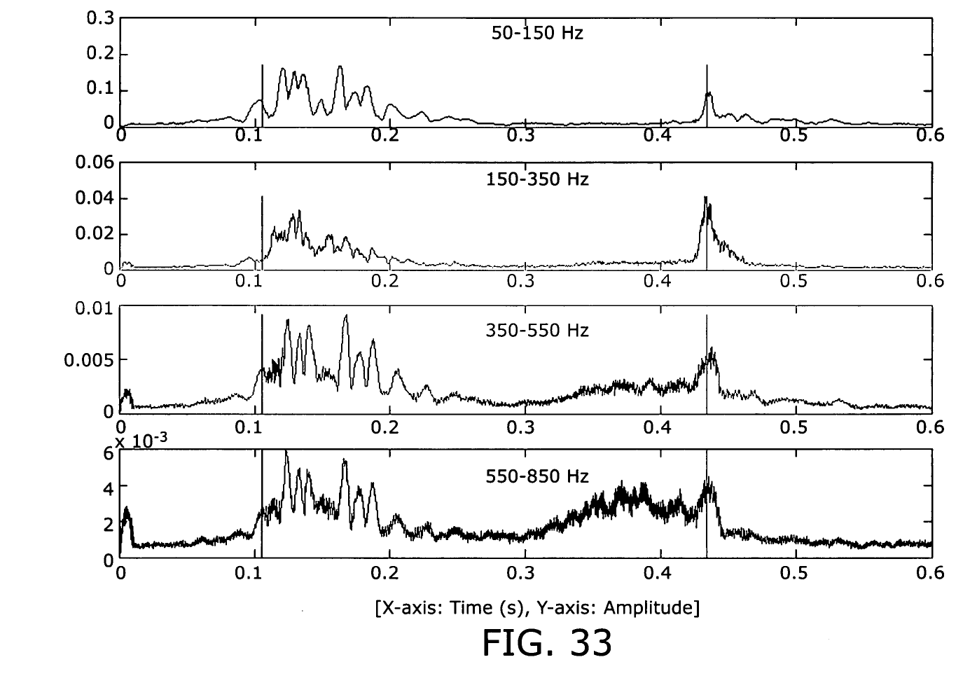

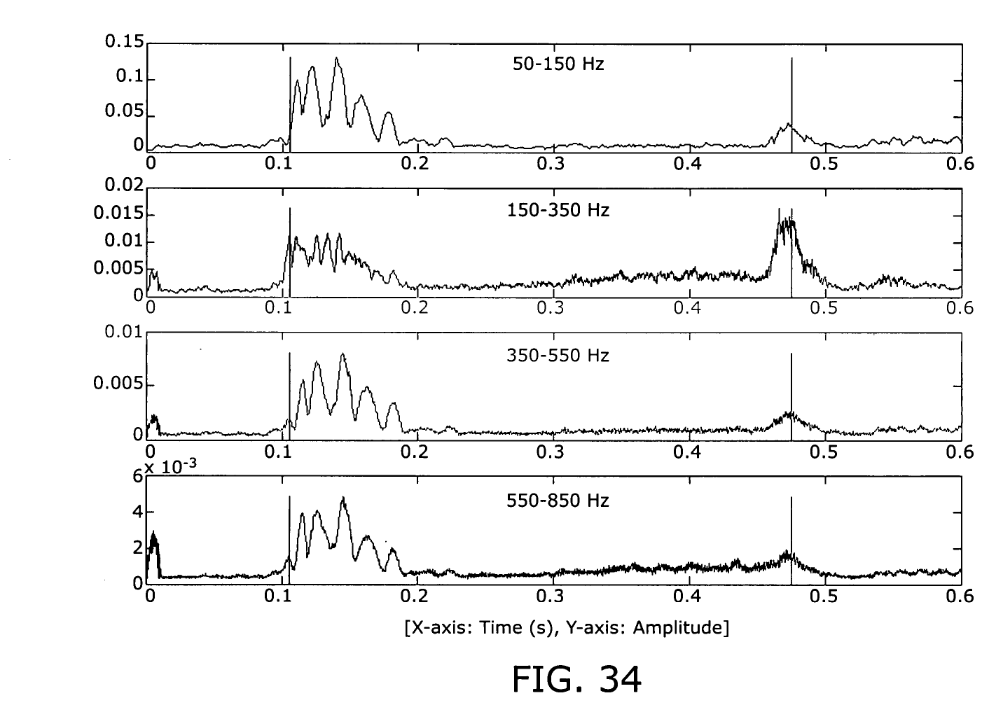

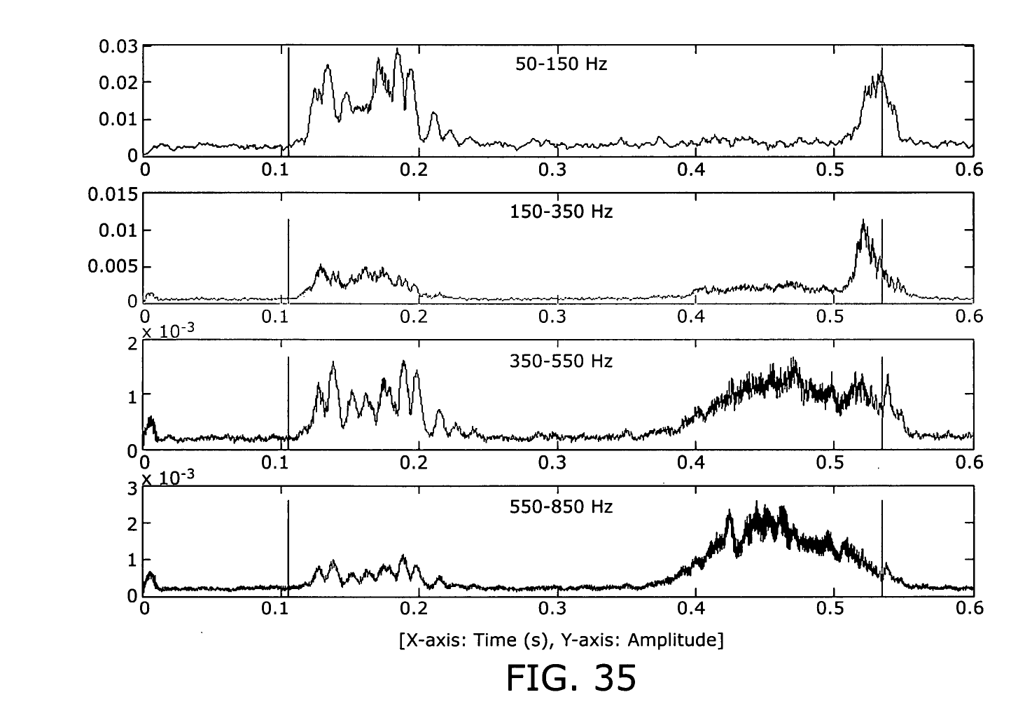

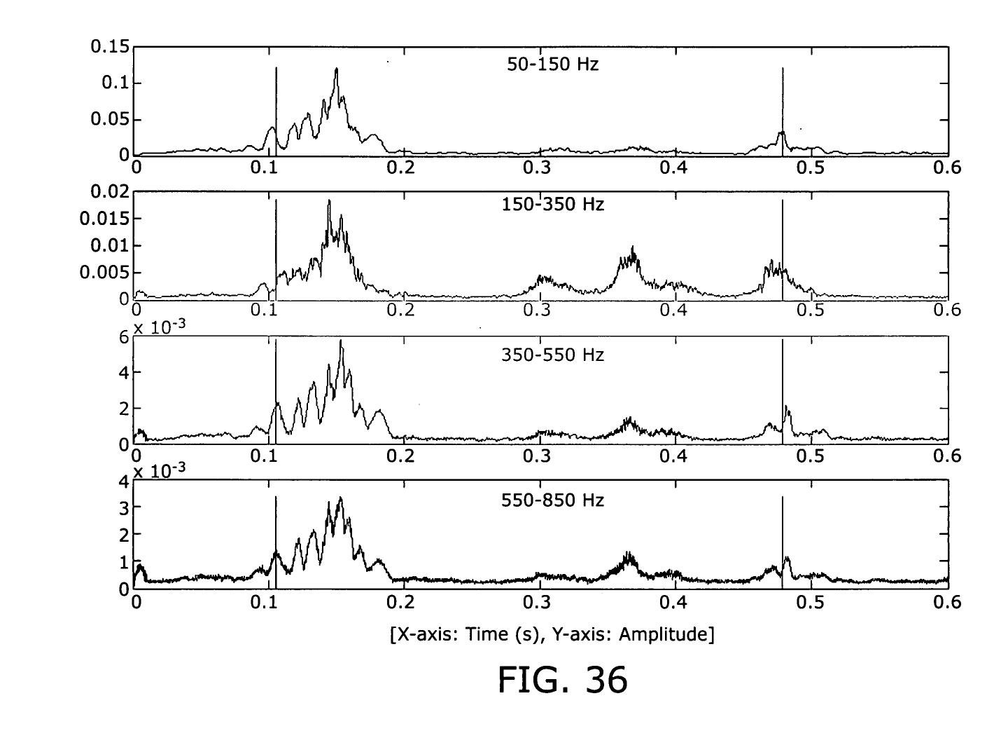

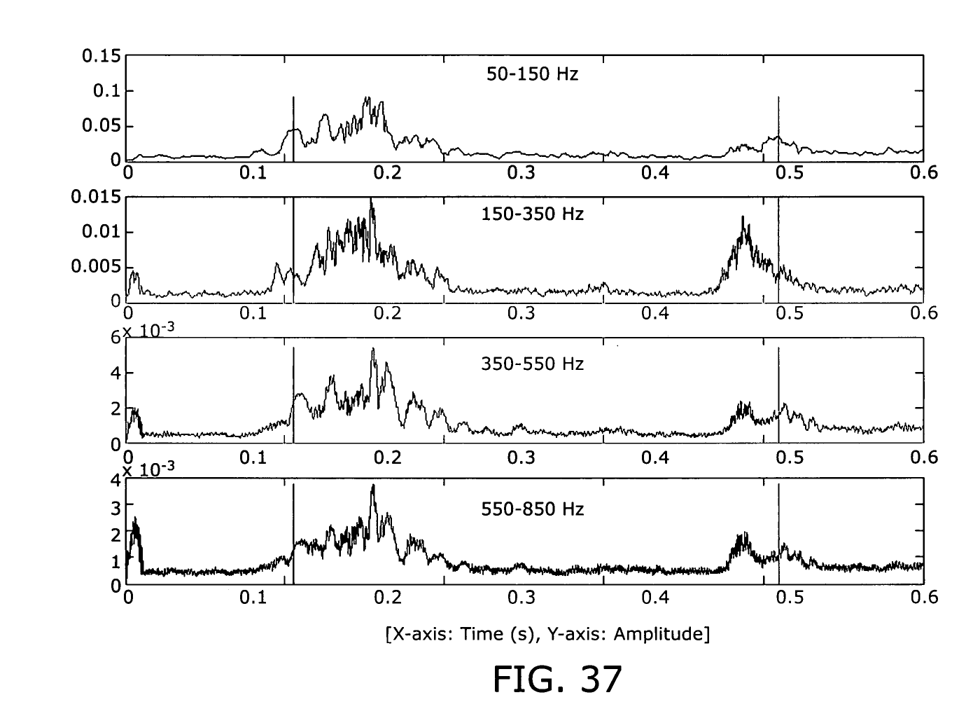

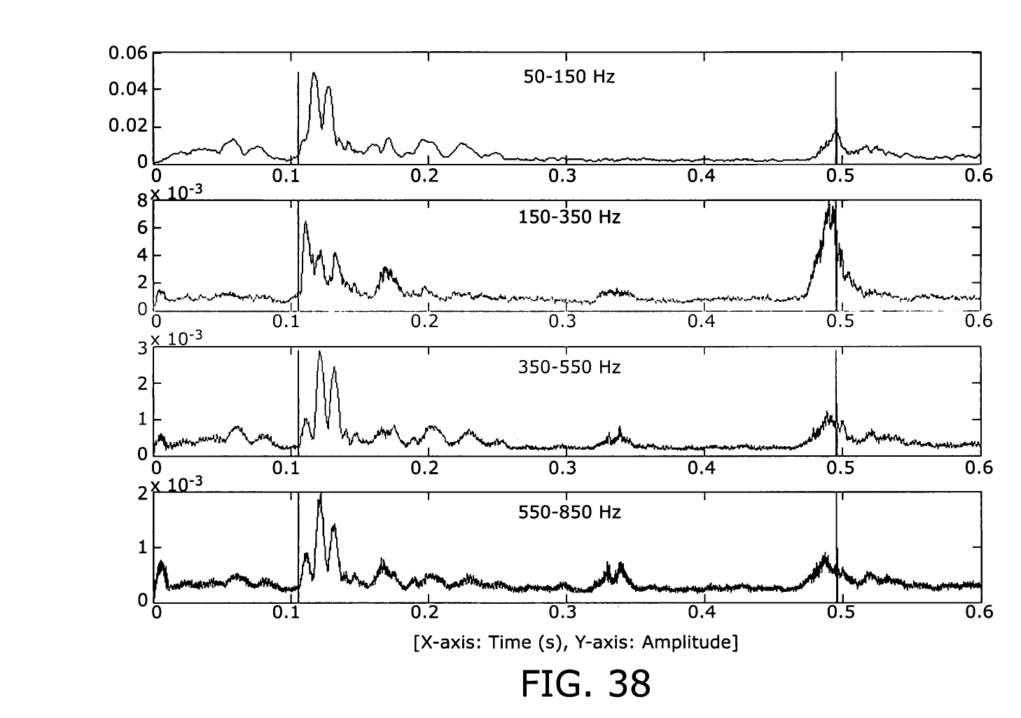

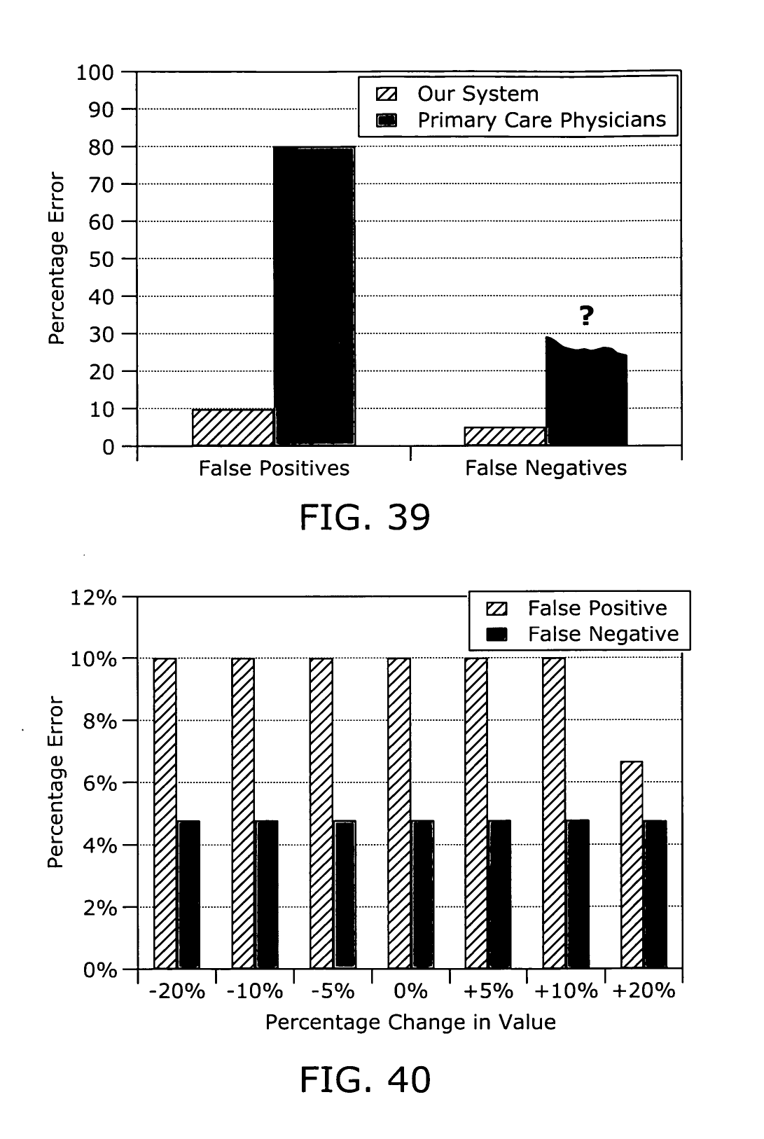

In the absence of MVP, the peaks in energy at higher frequencies are solely the result of harmonics associated with S2. As a result, for normal patients and those with benign murmurs, the maximum signal amplitude occurs at or very close to S2. FIGS. 27, 28, and 29 illustrate this effect. In these and subsequent figures the vertical black line at the left indicates the detected location of the QRS complex. The vertical line on the right corresponds to the predicted position of S2. In contrast, for patients suffering from MVP there is typically substantial energy content at higher frequencies during the last half of systole and the position of maximum signal amplitude tends to shift significantly prior to S2. (See FIGS. 30, 31, and 32). However, in a minority of subjects the peaks at higher frequencies become flatter and significantly wider, extending into systole (FIGS. 33, 34, and 35). In some cases, additional peaks may appear well before S2, as shown in FIGS. 36, 37, and 38.

As a result of these phenomena, in certain embodiments of the invention rather than simply examining the position of maximum signal amplitude (which may not distinguish normal patients and those with benign murmurs from subjects in whom the onset of MVP leads to flatter, wider peaks or the presence of additional peaks prior to S2), the method calculates the earliest point in the last half of systole where the signal amplitude first exceeds a predetermined percentage or fraction of the peak value. The predetermined value may be selected empirically, e.g., by examining the performance of the system on a representative set of acoustic signals using various percentages. For example, in one implementation of the system for diagnosis of MVP, a predetermined percentage of 60% was selected as optimal by examining all values from 25% to 95% in increments of five. Using this value as representative, for the j-th frequency band of the i-th beat, define 60 peakpos_{i,j} to be:

$$60 {peakpos}_{i , j} = first \text{-} index \text{-} true [ {X}_{i , j} ( \frac{length ( {X}_{i , j} )}{2} : length ( {X}_{i , j} ) ) \geq 0.6 {peakpos}_{i , j} ]$$

This parameter allows the measurement of how early on during the last half of systole the presence of considerable energy can be detected. This result can be used to compute the lag between the earliest occurrence of energy and S2 (i.e., the time interval by which energy at higher frequencies precedes S2) by:

60prec_{i,j}=length(X_{i,j})−60peakpos

where 60prec_{i,j} is the lag between the earliest occurrence of energy and S2 for the j-th frequency band of the i-th beat. Since the S1-S2 intervals can vary significantly between patients, this value may be scaled, e.g., by the duration of systole. Thus the final metric, 60precscaled_{i,j} is given by:

$$60 {precscaled}_{i , j} = \frac{60 {prec}_{i , j}}{systoliclength}$$

(2) Beat Classification

This metric is then employed to classify beats on a per-beat basis or to classify a prototypical beat. The classification is then used to reach a clinical conclusion or recommendation. It will be appreciated that the manner in which the metric is used to classify beats will vary depending on the metric.

In the case of MVP, to classify each individual beat, a threshold value, tⱼ, is defined for each frequency band j such that if 60precscaled_{i,j} is at least tⱼ, the beat is declared as being indicative of MVP. The i-th beat is declared as belonging to a subject suffering from MVP if, for any value of j:

60precscaled_{i,j}≧tⱼ

Thresholds for the bands were determined empirically to be:

150-350 Hz: −0.045

350-550 Hz: −0.045

550-850 Hz: −0.02

Since the 50-150 Hz band corresponds to low frequencies, not useful for diagnosis of MVP in the context of this embodiment of the invention, it was not considered.

If a beat is not classified as being characteristic of MVP, it is assumed to belong to a subject who is either normal or has a benign murmur. In other embodiments of the invention further analysis of these beats or other beats belonging to these subjects may be performed.

(3) Conclusion or Recommendation

Given the classification for each beat, an overall label of MVP can be assigned to any particular file containing an acoustic signal if a predetermined percentage of the selected beats are indicative of MVP. The predetermined percentage can be selected in a variety of ways depending on system goals for sensitivity and specificity, e.g., the degree to which the system is intended to tolerate false negatives and false positives. For example, a label of MVP can be assigned to a file if 40% of the selected beats are indicative of MVP. This value was chosen to minimize the error rate obtained on a per file basis while varying the parameter between 0% and 100% in increments of 5, using a file of acoustic signals recorded from a set of subjects either having or not having MVP (See Evaluation Section). A higher value would be expected to result in more false positives, while a lower value would be expected to result in more false negatives.

According to certain embodiments of the invention the system examines a plurality of files comprising acoustic signals for a subject. These recordings may correspond, for example, to different patient positions and signals acquired from different anatomic sites. As is known in the art, features of the acoustic signals characteristic of various conditions and diseases of the cardiovascular system may vary depending on patient position and recording site. The health care provider or other user of the system may supply it with relevant information such as the patient position or recording site, or such information may be part of the file itself and detectable by the system. The system may use this information to enhance performance. For example, where multiple files are evaluated, the system may base its conclusion on the file(s) in which features of the condition are expected to be more prominent. Different metrics may be used depending on the subject's position, recording site, etc. The system may employ a combination of metrics in order to classify a beat. In addition, the system may make use of clinical information (e.g., symptoms or signs of cardiovascular disease, patient history, etc.). Such information may be provided by the user or acquired automatically, e.g., from an electronic medical record.

In addition to, or instead of, classifying the individual beats, in certain embodiments of the invention the system classifies a prototypical beat and declares the subject to be suffering from a disease or condition if it meets either the criteria established for the individual beats, or a different criterion. The specific mode of operation can be determined by the user depending, for example, on the acceptable trade-off between noise reduction and the preservation of information related to the variation between beats. It is noted that variation in the length of the beats may contain useful information.

The conclusion or recommendation can be stated simply as whether or not the subject is predicted to have a particular disease or condition, or it can be expressed in terms of the likelihood that the subject suffers from the condition or disease. According to certain embodiments of the invention the system provide the users with a narrative explanation of the basis of the conclusion or recommendation. The recommendation or conclusion may be presented in conjunction with any of a number of audio-visual aids, which are further described below.

In addition to, or instead of, providing a diagnosis (e.g., whether or not a subject suffers from a disease or clinical condition, or the extent or severity of such condition), according to certain embodiments of the invention the system provides a variety of other recommendations. For example, the system may suggest additional diagnostic studies or may suggest referral to a specialist, e.g., if the system detects presence of a condition or is unable to determine whether or not a condition exists. The system may present a ranked list of possible conditions or diseases that the subject may have, which may include an estimate of the probability that the subject has a particular condition. Information such as the duration and/or intensity of murmurs may be presented, e.g., in accordance with standard rating systems.

The conclusion or recommendation, etc., may be output on a display, printed, inserted into an electronic medical record for the subject, entered into a database, etc. According to certain embodiments of the invention the system issues an evaluation similar to that which would be made by a physician, e.g., “MVP with mild, moderate, or severe regurgitation”.

III. Evaluation of the System

The representative embodiment of the invention described above for evaluation of subjects for mitral valve prolapse was evaluated using a dataset consisting of acoustic signals and simultaneously recorded EKGs for patients initially diagnosed with MVP by their primary care physicians and referred for additional diagnostic tests and family members of such patients. The dataset included fifty-one patients. Thirty of these have normal hearts or benign murmurs, while the remaining twenty-one suffer from MVP as diagnosed by echocardiogram. For each patient, two recordings were studied. Signals were collected from the apex and left lower sternal border with the patient lying down. All data was sampled at 44 kHz with 16-bit quantization. It will be appreciated that other parameters for digitization of the signal may be used.

As detailed below, the results indicate that the system performs significantly better than primary care physicians in diagnosing MVP and also suggest that the system can reduce the number of patients classified as being non-MVP when they do in fact suffer from the disease. Thus the system achieves both a reduction in false positive and false negative rates. The system achieves similar results using either individual beat classification or classification of a prototypical beat. In particular, the prototypical beat detection method performs better than the beat to beat detection method in terms of minimizing false positives, and both methods work identically for minimizing false negatives. Therefore, the results presented herein focus on prototypical beat detection, contrasting the results achieved by this approach to the classification rates obtained by primary care physicians.

The performance of the system is presented in Table 2 and FIG. 40.

TABLE 2

| | MVP | Non-MVP |
|---|---|---|
| Correctly diagnosed | 20 (95%) | 27 (90%) |
| Incorrectly diagnosed | 1 (5%) | 3 (10%) |

As shown therein, the system correctly diagnosed 95% of the patients that were deemed by echocardiography to suffer from MVP and correctly diagnosed 90% of the patients deemed not to suffer from MVP. Compared with primary care physicians, the system thus achieves a reduction in false positive rate from approximately 80% to 10%. The patients were evaluated by a trained cardiologist (F. Nesta) based on the degree of MVP heard during auscultation. Twenty-one percent of the patients suffering from MVP as assessed by echocardiography were assigned the same scores as non-MVP patients. In contrast, the automated auscultation system obtained only a single false negative, corresponding to a patient with minimal MVP with no regurgitation, suggesting that the system achieves a lower false negative rate than achievable by primary care physicians and trained specialists.

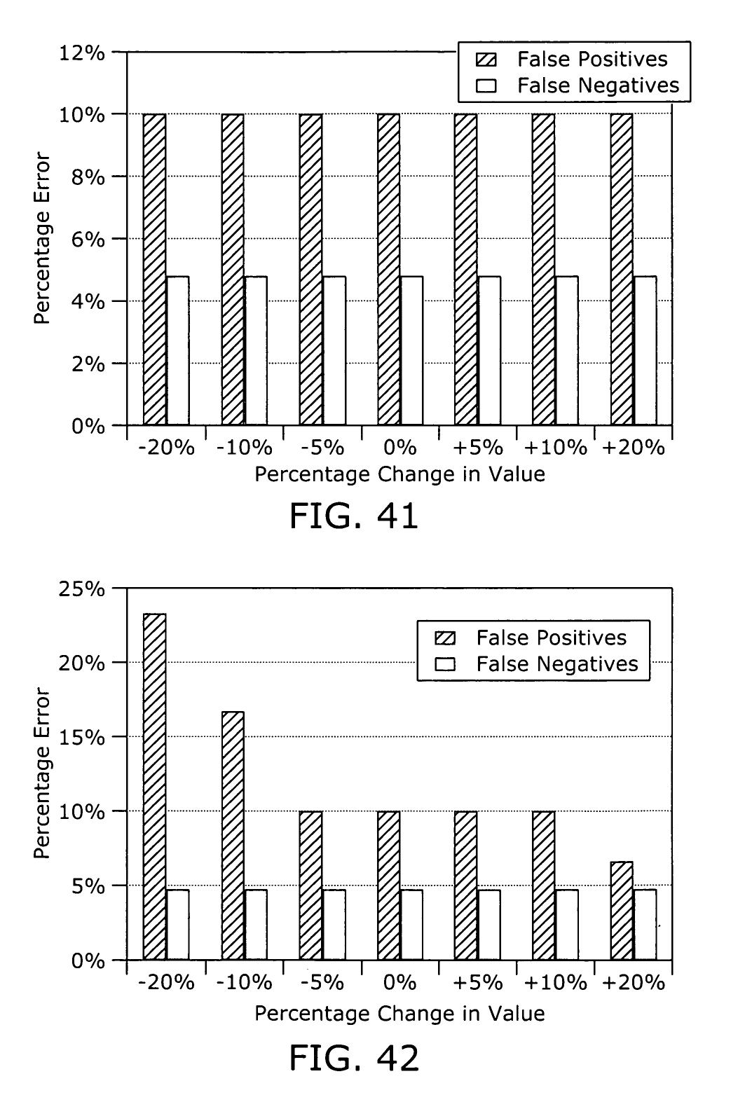

FIGS. 40-42 show how the false positive and false negative rates are affected by changes to the thresholds chosen for the high frequency bands. As can be seen, small changes do not lead to significantly different diagnoses. It appears that performance of the system would be improved further by increasing the thresholds for the 150-350 Hz and 550-850 Hz bands. This appears to reduce false positives while keeping false negatives constant. However, for a larger dataset an increase in the threshold values may lead to a corresponding increase in the false negative rate, which may not be an acceptable trade-off for the decrease in false positives, particularly when the system is used for screening purposes.

IV. Audio-Visual Diagnostic Aids

According to certain embodiments of the automated auscultation system, a variety of audio-visual diagnostic aids are also provided. In certain embodiments of the invention these aids are integrated with the system in the sense that at least a portion of the information presented by the aids reflects the actual information used by the system in arriving at a conclusion or recommendation, and the aids illuminate the process by which the system arrives at the conclusion or recommendation, making it more understandable to the health care provider and facilitating learning. These aids and their methods of operation are discussed below. The aids are of use both individually and as a group. The aids are of use both in the context of automated diagnosis and with those embodiments of the system in which no conclusion or recommendation is provided.

A. Prototypical Heart Beat Visualization

Visualization of the prototypical beat provides an effective means of visualizing the diagnostic information contained in the acoustic signal. While it may also be useful to display individual beats, if these beats are displayed sequentially it may be difficult to compare non-adjacent beats, rendering inter-beat comparisons difficult. Displaying multiple beats simultaneously (particularly displaying their time-frequency decomposition as described below) can lead to information overload. Thus according to certain preferred embodiments of the invention the system displays a prototypical beat instead of, or in addition to, individual beats.

In certain embodiments of the invention different frequency bands are displayed individually in order that features of the energy distribution characteristic of conditions or diseases may be more readily visualized. The band-aggregated result of the time frequency analysis described above may be plotted directly to a display device, thereby achieving the desired separation while avoiding the need for redundant computation.

The bands may be individually scaled in order to emphasize features of diagnostic importance. For example, as described above, MVP is characterized by increased energy in the higher frequency bands during the second half of systole, relative to the energy distribution in normal subjects. However, the characteristic MVP signature has significantly lower energy content relative to the remainder of the signal, so information at the frequencies of interest is typically obscured by heart sounds at lower frequencies. Scaling addresses this problem. In addition to scaling the axis corresponding to energy, it also possible to scale the time axis. This allows the stretching out of events that may otherwise be difficult to hear or see, e.g., because they are very short.

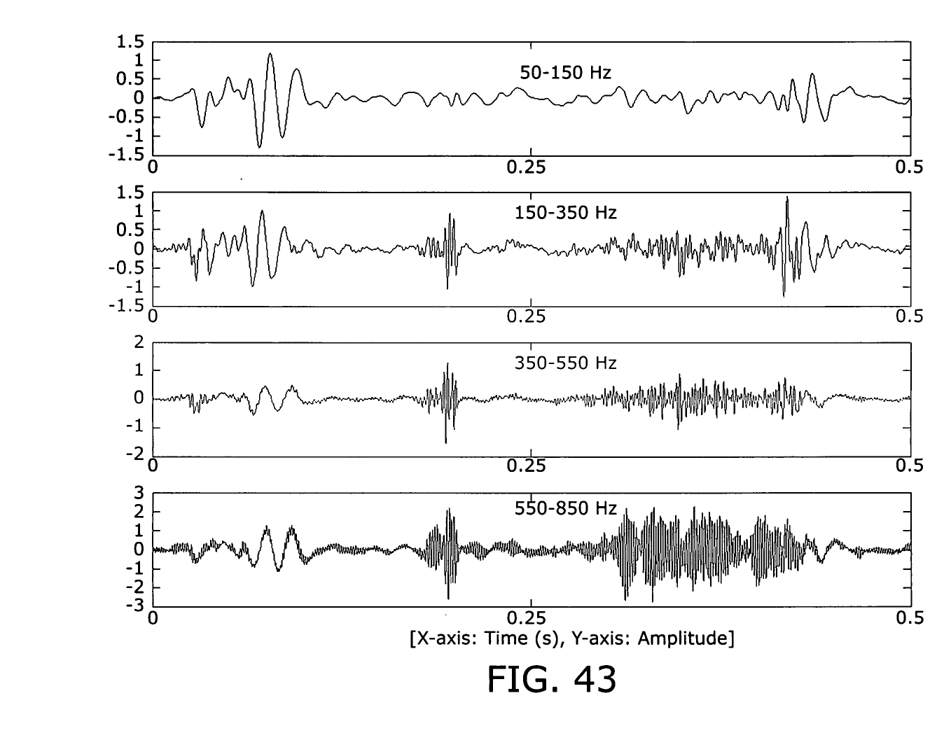

Events that are close together can be distinguished by providing a fixed “snapshot” of an entire beat at one time. This allows the position of two or more events, and the interval between them, to be examined in detail for any given beat. Viewing entire beats allows comparisons in morphology, amplitude, location, etc., to be conducted between different parts of the signal. Whereas listening presents only a fraction of the total information in the beat at any time, a visual display (such as that presented in FIG. 43) can be used to output the content of the signal in its entirety, allowing the separation of various events in both time and frequency to be observed.

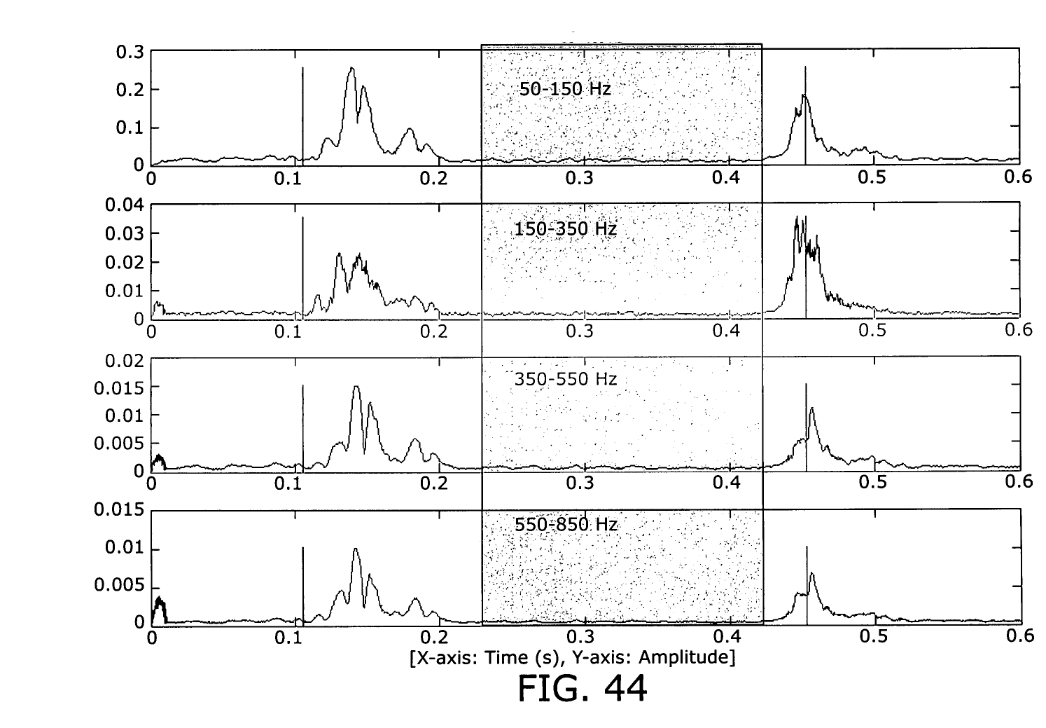

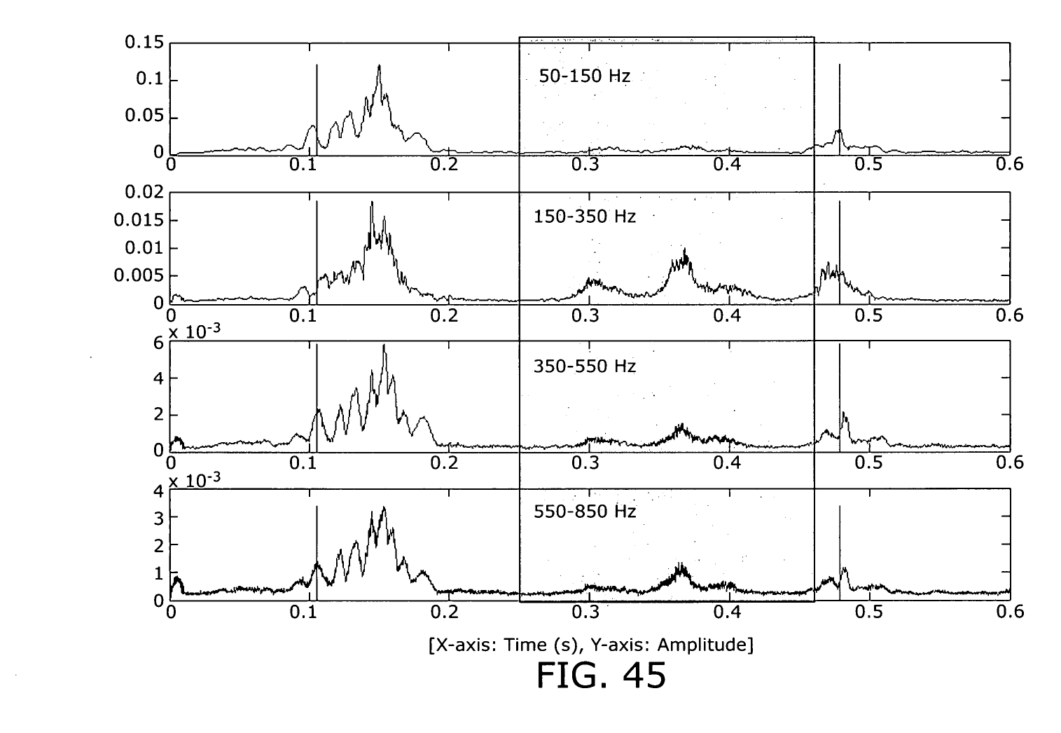

In one embodiment of the invention, the visual aid plots the four frequency bands of a beat to a screen as shown in FIGS. 44 (non-MVP subject) and 45 (MVP subject). In addition to plotting the four bands, the positions of the QRS complex (vertical line at the left) and S2 (vertical line at the right) are displayed, and the region of interest in the signal for detecting MVP is highlighted. Similar highlighting may be performed for other cardiac events and for regions of interest in the evaluation of other conditions. The plots illustrate the relative ease with which MVP can be identified visually according to the inventive methods even though this might not be the case by listening to the recorded heart sounds. Sound files for these subjects are provided on the web at http://maas.lcs.mit.edu/sounds/. The patient shown in FIG. 44 corresponds to the file labeled “Normal” on the website, whereas the “Tricky MVP” file presents the signal recorded for the patient in FIG. 45.

(B) Reduced Rate Playback with Preservation of Frequency Content

A second audio-visual aid provides the ability to play back heart sounds at reduced rates. This facilitates differentiating between acoustical events by making the separation between them more noticeable.

One possible approach to achieve reduced rate playback is to upsample the recorded signal and play it back at the same sampling rate at which it was initially recorded. However, this technique modifies the frequency content of the signal [25]. In the case of a condition such as MVP characterized by energy at higher frequencies, alternate approaches are preferable. In general, a variety of approaches to altering the speed of playback without altering the frequency content of a signal are known in the art [26, 27, 28, 29, 30, 31, 32]. The present invention encompasses the recognition that certain of these techniques and others may be used to play back acoustic signals emanating from the cardiovascular system at an altered (e.g., reduced) rate without altering the frequency content, as well as the recognition that such playback provides a useful diagnostic and learning tool. In order to achieve reduced rate playback of acoustic signals emanating from the cardiovascular system, one embodiment of the invention employs the phase vocoder implementation for timescale modification described in [32], which is open-source based an coded in Matlab, facilitating incorporation into the system. A sample of the slowed down heart sounds produced by this approach is found on the web at http://maas.lcs.mit.edu/sounds/.

(C) Enhanced Audio-Prototypical Beat Playback

In certain embodiments of the invention the system allows for playback of a slightly modified version of the prototypical beat. Since the time-envelope characterization described above leads to a signal that has a positive value at every time instant and changes the auditory characteristics of the signal, a slightly imprecise version of the prototypical beat is constructed that does not include this step. The rest of the procedure for constructing the prototypical beat is unchanged, and the resulting signal is referred to herein as the audio-prototypical beat.

The audio-prototypical beat can be reconstituted from its different frequency components by adding these components together. The components may be normalized as described above prior to combination. Although removing the time-characterization step during construction of the audio-prototypical beat may lead to destructive interference, this loss of information is normally insignificant, and subjectively the audio-prototypical beat makes it considerably easier to hear murmurs due to MVP.

A sample of the enhanced audio-prototypical beats produced by the approach described above can be found on the web at http://maas.lcs.mit.edu/sounds. A variety of other methods may be used to enhance the audio playback of the individual beats and/or the prototypical beat.

D. Other Audio-Visual Components and Features

Any of a variety of additional audio-visual components may be included. For example, the system may display a standard EKG, which may be aligned with the prototypical beat. Any of the displays can be annotated, e.g., to show significant cardiac events such as S1, S2, and significant features of the EKG and/or acoustic signal (e.g., features indicative of existence of abnormal conditions such as murmurs). The audio playback can also be annotated, e.g., with “beeps” to identify significant features. The audio playback may play back the differential between signals recorded simultaneously, e.g., from different regions of the body. The display may allow zooming in and out. A variety of statistical metrics may be presented, e.g., a histogram showing variance of beat length or other features. In certain embodiments of the system files containing examples of acoustic signals and prototypical beat representative of conditions and diseases of the cardiovascular system are included. These files may be used for teaching and comparison purposes.

V. Additional Implementation Details

As mentioned above, the invention is preferably implemented in software but may be implemented in various forms of hardware, software, firmware, special purpose processors, or combinations of any of these.

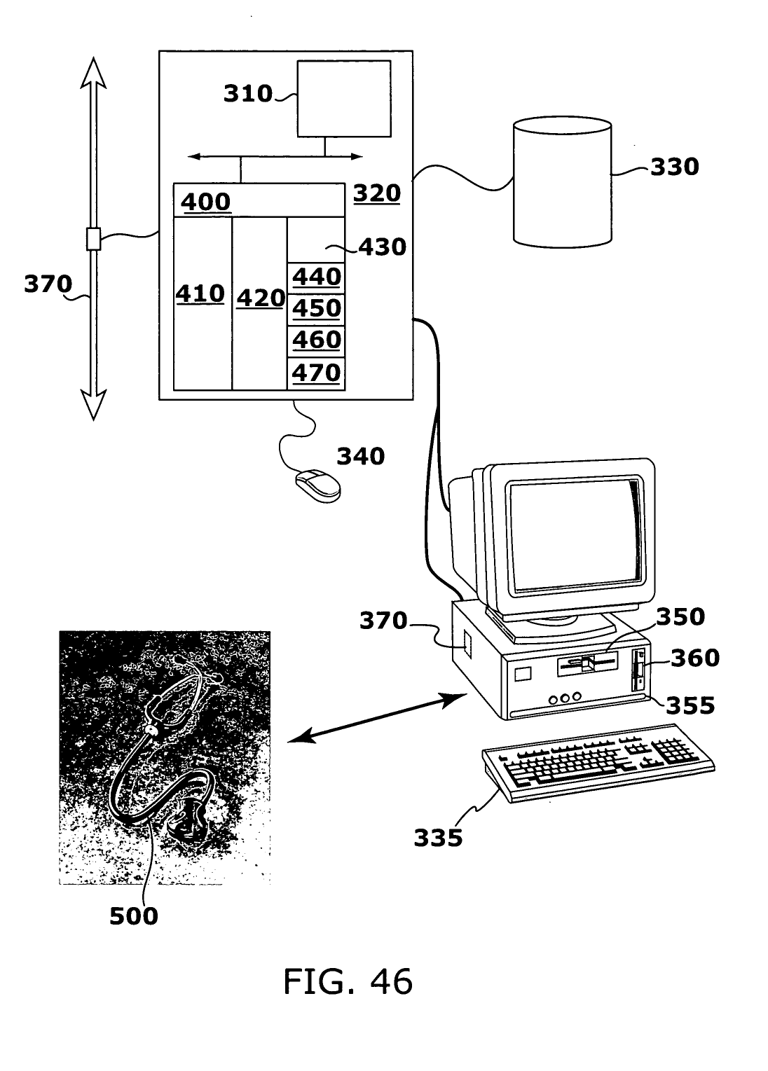

In preferred embodiments the present invention includes, or is used in conjunction with, a computer or similar data processing device for analyzing the acoustic and/or electrocardiographic signals, generating a clinical conclusion or recommendation, and processing the signals for purposes of the audio-visual aids as described above. FIG. 46 depicts a representative embodiment of a computer system and electronic stethoscope that may be used for this purpose. Computer system 300 comprises a number of internal components and is also linked to external components. The internal components include processor element 310 interconnected with main memory 320. For example, computer system 310 can be a Intel Pentium™-based processor such as are typically found in modern personal computer systems. The external components include mass storage 330, which can be, e.g., one or more hard disks. Additional external components include user interface device 335, which can be a keyboard and a monitor including a display screen, together with pointing device 340, such as a “mouse”, or other graphic input device. The interface allows the user to interact with the computer system, e.g., to cause the execution of particular application programs, to enter inputs such as data and instructions, to receive output, etc. The computer system may further include disk drive 350, CD and/or DVD drive 355, and zip disk drive 360 for reading and/or writing information from or to floppy disk, CD, DVD, or zip disk respectively. Preferably the computing device is equipped with a sound card 370 for digitization of the acoustic signal, although digitization may also be accomplished elsewhere. It is noted that the preceding description is for representative purposes only and that many of the devices mentioned are optional.

The computer system is typically connected to one or more network lines or connections 370, which can be part of a network link to other local computer systems, remote computer systems, or wide area communication networks, such as the Internet. This network link allows computer system 300 to share data and processing tasks with other computer systems and to communicate with remotely located users. The computer system may also include components such as a display screen, printer, etc., for presenting information, e.g., for displaying the visual components of the suite of audio-visual aids.

A variety of software components, which are typically stored on mass storage 330, will generally be loaded into memory during operation of the inventive system. These components function in concert to implement the methods described herein. The software components include operating system 400, which manages the operation of computer system 300 and its network connections. This operating system can be, e.g., a Microsoft Windows™ operating system such as Windows 98, Windows 2000, or Windows NT, a Macintosh operating system, a Unix or Linux operating system, an OS/2 or MS/DOS operating system, etc. Software component 410 is intended to embody various languages and functions present on the system to enable execution of application programs that implement the inventive methods. Such components, include, for example, language-specific compilers, interpreters, and the like. Any of a wide variety of programming languages may be used to code the methods of the invention, including microcode and high-level programming languages. Such languages include, but are not limited to, C, C++, JAVA™, various languages suitable for development of rule-based expert systems such as are well known in the field of artificial intelligence, software packages such as Matlab® that provide signal processing routines, etc. According to certain embodiments of the invention the software components include Web browser 420, e.g., Internet Explorer™ or Netscape Navigator™ for interacting with the World Wide Web.

Software component 430 represents the software components of the invention, e.g., the beat selection component, the time-frequency analysis component, and the processing component that implements the decision mechanism to provide a clinical conclusion or recommendation. Software component 440 represents the audio-visual aids described above. The computer system also includes database 450, which may comprise patient medical records, and database 460, which may be used to store audio and video files for future playback, etc. Database 470 includes a library of acoustic signal files and accompanying prototypical beats illustrative of various conditions and diseases that the system is capable of evaluating, which may be annotated to facilitate their use for teaching purposes. Computer system 300 interfaces with electronic stethoscope 500.

Software components of the invention may be provided in the form of computer-executable instructions (code) stored on a computer-readable medium such as a floppy disk, CD, DVD, zip disk, or the like. The software may also be downloaded, e.g., from the Internet, in which case it is transferred electronically, e.g., directly to the user's computer, where it may be stored.

As mentioned above, the automated auscultation system may be implemented using an electronic stethoscope to acquire the acoustic and/or EKG signals. A variety of suitable stethoscopes are known in the art. See, e.g., U.S. Ser. Nos. 6,134,331; 6,295,365; 6,512,830, 6,533,736 (disclosing a wireless electronic stethoscope) and references in the foregoing patents, all of which are included herein by reference. Suitable electronic stethoscopes are available, for example, from Meditron, Inc., Vettre, Norway (http://www.meditron.no), which may be connected to a computer. In general, such stethoscopes comprise a microphone including a sensor for detecting acoustic signals emanating from the cardiovascular system. Preferred sensors display good sensitivity in the frequency range up to at least 900 Hz, which includes those frequencies of interest in the evaluation of conditions of the cardiovascular system as well as frequencies characteristic of breath sounds. See, e.g., U.S. Published patent applications 20010014162 and 20030093003 and [56] in addition to the patents mentioned above. For evaluation of conditions such as MVP in which diagnostic information is contained in the higher frequency bands, it is important that the sensor display good sensitivity in the upper portion of the 0-900 Hz band. Methods of acquiring the signal that do not involve use of an electronic stethoscope may also be employed provided that they include an appropriate sensor. In general, the signal may be preamplified and/or filtered prior to digitization and subsequent analysis according to the methods of the invention.

In certain embodiments of the invention the electronic stethoscope is equipped with EKG leads, which are also connected to the computer. Other methods of acquiring an EKG signal may also be used. In general, the acoustic and/or EKG signals may be transmitted to the computer wirelessly.

Software components of the invention may be supplied together with an electronic stethoscope and any necessary additional components, e.g., EKG leads, connectors, etc.

The description above has generally envisioned a system in which the user interacts directly with the computer that executes the application program encoding the methods of the invention. However, according to certain embodiments of the invention the system is implemented as a client/server system in which signal is entered and then transmitted to a server computer that analyzes the signal and generates the conclusion or recommendation, which is then transmitted to the client system. The client computer system can comprise any available computer but is typically a personal computer equipped with a processor, memory, display, keyboard, mouse, storage devices, appropriate interfaces for these components, and one or more network connections. The server computer system typically includes most or all of the same components, but may, for example, have a more powerful processor.

The foregoing description is to be understood as being representative only and is not intended to be limiting. Alternative systems and techniques for implementing the methods of the invention will be apparent to one of skill in the art and are intended to be included within the accompanying claims.

### References

[1] Pease, A. “If the Heart Could Speak”, Pictures of the Future, Fall 2001, Seimens Webzine (http://w4.siemens.de/FuI/en/archiv/pof/heft2 01/artikel19/)

[2] Craige, E. “Should Auscultation be Rehabilitated?”, New England Journal of Medicine 1988, 318:1611-3.

[3] Mangione et al., “Cardiac Auscultatory of Internal Medicine and Family Practice Trainees: a Comparison of Diagnostic Proficiency”, JAMA 1997;278:717-722.

[4] Mangione et al, “The Teaching and Practice of Cardiac Auscultation During Internal Medicine and Cardiology Training: a Nationwide Survey”, Ann Intern Med 1993;1 19;47-54.

[5] “Facts about Mitral Valve Prolpase”, National Heart, Lung and Blood Institute Information Booklet. (http://www.nhlbi.nih.gov/health/public/heart/other/mvp/mvp fs.htm)

[6] Hoffman, R. “Mitral Valve Prolapse”, Conscious Choice (www.consciouschoice.com/holisticmd/hmd093.html)

[7] MEDLINEplus Medical Encyclopedia: Mitral Valve Prolapse (http://www.nlm.nih.gov/medlineplus/ency/article/000180.htm)

[8] The Physician and Sportsmedicine: Mitral Valve Prolapse (http://www.physsportsmed.com/issues/1996/07 96/joy.htm)

[9] Heart Sounds and Murmurs (http://www.mcevoy.demon.co.uk/Medicine/Medicine/ClinExamn/Murmurs.html)

[10] Heart Murmurs and Other Abnormal Heart Sounds, NurSpeak (http://www.nurspeak.com/tools/murmurs.htm)

[11] Bates, B. “A Guide to Physical Examination”, 4th edition. J. B. Lippincott Co., Philadelphia 1987.

[12] Yancone-Morton, L. “Cardiac assessment”, RN 54:(12)28-35, December 1991.

[13] Valsalva Maneuver, HealthAtoZ http://www.healthatoz.com/healthatoz/Atoz/ency/valsalva maneuver.html

[14] Dr. Robert Levine, Personal Communication.

[15] MATLAB is a trademark of The Mathworks, Inc., 3 Apple Hill Drive, Natick, Mass. 01760-2098, USA. (http://www.mathworks.com/)

[16] Friesen, G. et al, “A comparison of the noise sensitivity of nine QRS detection algorithms”, IEEE Transactions on Biomedical Engineering, Vol. 37, No. 1, January 1990

[17] Fraden, J. et al, “QRS Wave Detection”, Med. Biol. Eng. Comput., vol 18, pp.125-132, 1980.

[18] Dr. Nathaniel Sims, Personal Communication.

[19] Dr. Francesca Nesta, Personal Communication.

[20] Oppenheim, A. et al, “Discrete-Time Signal Processing”, Prentice Hall Signal Processing Series

[21] Lim, J., “Two-Dimensional Signal and Image Processing”, Prentice Hall, 1990.

[22] Gonzalez, R. et al, “Digital Image Processing” Addison-Wesley, 1993.

[23] Borda, R. et al, “Error reduction in small sample averaging through the use of the median rather than the mean”, Electroenceph Clin Neurophysiol, v. 25 (1968), pp. 391-392.

[24] Tukey, J. “Exploratory Data Analysis” Reading, Mass.: Addison-Wesley, 1971.

[25] Oppenheim, A. et al, “Signals and Systems”, Prentice Hall, 1996

[26] Fairbanks, G. et al, “Method for time or frequency compressionexpansion of speech”, IEEE Trans. Audio and Electroacoustics, AU-2, pp. 712, 1954.

[27] Flanagan, J. et al “Phase Vocoder”, Bell System Technical Journal, November 1966, 1493-1509. (http://www.ee.columbia.edu/dpwe/e6820/papers/FlanG66.pdf)

[28] Lee, F., “Time compression and expansion of speech by the sampling method”, J. Audio Eng. Soc., Vol. 20:3, pp. 738742, 1972.

[29] Dolson, M., “The phase vocoder: A tutorial”, Computer Music Journal, vol. 10, no. 4, pp. 14 - 27, 1986.

[30] Suzuki, R. et al, “Time-scale modification of speech signals using crosscorrelation functions”, IEEE Transactions on Consumer Electronics, Vol. 38:3, pp. 357363, 1992.

[31] Laroche, J., “New Phase Vocoder Technique for Pitch-Shifting, Harmonizing and Other Exotic Effects”, IEEE Workshop on Applications of Signal Processing to Audio and Acoustics. Mohonk, New Paltz, N.Y. 1999. (http://www.ee.columbia.edu/dpwe/papers/LaroD99-pvoc.pdf)

[32] Ellis, D., “A Phase Vocoder in MATLAB”, Columbia University (http://www.ee.columbia.edu/dpwe/resources/matlab/pvoc/)

[33] Cathers, I. “Neural network assisted cardiac auscultation”, Artif Intell Med. 1995;7:5366.

[34] Guo, Z. et al. “Artificial neural networks in computer assisted classification of heart sounds in patients with porcine bioprosthetic valves”, Med Biol Eng Comp. 1994;32:311316.

[35] Coehn, M. et al. “Comparative Approaches to Medical Reasoning”, River Edge, N.J.: World Scientific; 1995:271288.

[36] Okuni, M. et al, “Trial of a new cardiac mass screening system in school children”, Jpn Circ J. 1978;42:4952.

[37] Reynolds, J. “Heart disease screening of preschool children”, Am J Dis Child. 1970;119:488493.

[38] White, P. et al, “Time frequency analysis of heart murmurs in children” Proc IEEE. 1997;3:14.

[39] El-Asir, B. et al, “Time frequency analysis of heart sounds”. Proc IEEE. 1996;12:553558

[40] Leung, T. et al, “Analysing pediatric heart murmurs with discriminant analysis”, Proc IEEE. 1998;18:16281631.

[41] Sarkady, A. et al, “Computer analysis techniques for phonocardiogram diagnosis”, Comput Biomed Res. 1976;9:349363.

[42] DeGroff, C. et al, “Artificial Neural NetworkBased Method of Screening Heart Murmurs in Children”, Circulation 2001;103:2711-2716.

[43] Winston, P. “Artificial Intelligence”, Addison-Wesley Pub Co, 3rd edition, 1992.

[44] Dewan, S. et al, “Heart Murmurs”, Good Health Magazine, February/March 1998. (http://www.seton.net/Wellness/GoodHealthMagaine/GoodHealthMagaineArOA67/-FebMarBJJILoveYourH09829/HeartMurmurs.asp)

[45] Shino, H. et al, “Detection and classification of systolic murmur for phonocardiogram screening”, Proc. of the 18th Intl Conf. of the IEEE Eng. in Med. and Biol. Soc., Volume 1, pp. 123124. Amsterdam, The Netherlands.

[46] Barschdorff, D. et al, “Automatic phonocardiogram signal analysis in infants based on wavelet transforms and artificial neural networks”, Computers in Cardiology 1995, pp. 753-756. IEEE, Vienna, Austria.

[47] Reed, T. et al, “The Analysis of Heart Sounds for Symptom Detection and Machine-Aided Diagnosis”, 2nd Conference on Modelling and Simulation in Biology, Medicine and Biomedical Engineering, Delft, The Netherlands

[48] Watrous, R. et al, “Wavelet based bank of correlators approach for phonocardiogram signal classification”, Proc. of the IEEE-SP Intl Symp. on Time-Frequency and Time-Scale Analysis, pp. 7780. Pittsburgh, Pa.

[49] Myint, W. et al, “An Electronic stethoscope with diagnosis capability”

[50] Banerji, A. et al. “Circulation: Cardiovascular System”, Life Science Discipline, LA Mission College (http://207.233.44.253/wms/reynolmj/lifesciences/lecturenote/bio3/Chap23.ppt)

[51] Gulaff, E. “Cardiac Cycle”, Perfusion Education, KFSH&RC (http://www.kfshrc.edu.sa/perfusion/education/CardiacCycle.ppt)

[52] Fisher, J. “Cardio-Vascular Physiology”, Queen's University Faculty of Health Sciences (http://meds.queensu.ca/medicine/physiol/undergrad/phase2/Lecture5 f.ppt)

[53] “The ECG”, Cardionetics (http://www.cardionetics.com/docs/healthcr/ecg.htm) λ54] The Auscultation Assistant, Physiology (http://www.wilkes.med.ucla.edu/Physiology.htm

[55] Widrow, B., et al., Adaptive Signal Processing, Prentice-Hall, 1985.

[56] Padmanabhan et al., in “Accelerometer Type Cardiac Transducer for Detection of Low-level Heart Sounds”, IEEE Transactions on Biomedical Engineering, 40(1), pp. 21-28, January 1993.

[57] Braunwald, E., Zipes, D., and Libby, P. Heart Disease: A Textbook of Cardiovascular Medicine, (2 volumes), 6^{th} ed., W. B. Saunders, 2001.

1. A method of performing automated auscultation of the cardiovascular system, the method comprising steps of:
(a) selecting one or more beats for analysis, wherein each beat comprises an acoustic signal emanating from the cardiovascular system;
(b) performing a time-frequency analysis of beats selected for analysis so as to provide information regarding the distribution of energy, the relative distribution of energy, or both, over different frequency ranges at one or more points in the cardiac cycle; and
(c) processing the information to reach a clinically relevant conclusion or recommendation; and
(d) presenting some or all of the information in a manner that facilitates a mechanistically comprehensible explanation of the basis for the conclusion or recommendation or how the conclusion or recommendation was reached.

2. The method of claim 1, further comprising:
providing a mechanistically comprehensible explanation of the basis for the conclusion or recommendation or of how the conclusion or recommendation was reached.

3. The method of claim 1, wherein the step of selecting one or more beats for analysis is performed automatically.

4. The method of claim 1, wherein more than one beat is selected for analysis.

5. The method of claim 1, wherein performing a time-frequency analysis comprises performing a time-frequency decomposition.

6. The method of claim 1, further comprising the step of:
combining information obtained from a plurality of the beats selected for analysis prior to the step of processing the information to reach a clinically relevant conclusion or recommendation.

7. The method of claim 1, further comprising the step of:
outputting the clinical conclusion or recommendation together with one or more of the following: a display of at least a portion of the acoustic signal, a display of an annotated or unannotated EKG tracing, a display of a frequency decomposed acoustic signal representing one or more beats, one or more enhanced beats, or a prototypical beat, and an audio playback of one or more beats or a prototypical beat.

8. The method of claim 7, wherein the clinical conclusion or recommendation is selected from the group consisting of: whether a pathological condition exists, whether a benign condition exists, whether to make a referral to a specialist, whether to perform an additional diagnostic study, and whether to initiate or alter therapy.

9. A method of performing automated auscultation of the cardiovascular system, the method comprising steps of:
(a) selecting one or more beats for analysis, wherein each beat comprises an acoustic signal emanating from the cardiovascular system;
(b) performing a time-frequency analysis of beats selected for analysis so as to provide information regarding the distribution of energy, the relative distribution of energy, or both, over different frequency ranges at one or more points in the cardiac cycle;
(c) combining information obtained from a plurality of the beats selected for analysis; and
(d) processing the information to reach a clinically relevant conclusion or recommendation,
wherein the step of performing a time-frequency analysis, the processing step, or both make use of knowledge of the physiological or mechanistic basis of a disease or clinical condition of the cardiovascular system.

10. The method of claim 9, wherein performing a time-frequency analysis comprises performing a time-frequency decomposition.

11. A method of performing automated auscultation of the cardiovascular system of a subject, the method comprising steps of:
(a) selecting one or more beats for analysis, wherein each beat comprises an acoustic signal emanating from the cardiovascular system;
(b) performing a time-frequency analysis of beats selected for analysis so as to provide information regarding the distribution of energy, the relative distribution of energy, or both, over different frequency ranges at one or more points in the cardiac cycle;
(c) if more than one beat was selected in step (a), combining information obtained from a plurality of the beats selected for analysis; and
(d) processing the information to reach a clinically relevant conclusion or recommendation.

12. The method of claim 11, wherein more than one beat is selected.

13. The method of claim 11, wherein the one or more beats are selected automatically.

14. The method of claim 11, wherein the step of selecting one or more beats for analysis comprises:
segmenting the acoustic signal representing a beat into regions of interest by locating the position of at least the second heart sound within the acoustic signal, wherein the second heart sound associated with that beat is located by searching over a predetermined time interval following the peak in the T wave for that beat and declaring the position of the second heart sound to be at the position of the peak in the acoustic signal within the predetermined time interval.

15. The method of claim 14, wherein the T wave for a beat is declared to be at the position of the peak in the EKG signal within an interval beginning a predetermined period of time following the QRS complex for that beat and within a predetermined fraction of the cardiac cycle immediately following the QRS complex for that beat.

16. The method of claim 15, wherein the second heart sound associated with a beat is located by searching over a predetermined time interval following the peak in the T wave for the beat and declaring the position of the second heart sound for the beat to be at the position of the peak in the acoustic signal within the predetermined time interval.

17. The method of claim 15, wherein the second heart sound associated with a beat is located by:
(a) initially searching over a predetermined time interval following the peak in the T wave for each of a plurality of beats and declaring the position of the second heart sound for each beat to be at the position of the peak in the acoustic signal within the predetermined time interval;
(b) computing the systolic length for the plurality of beats using the positions of the second heart sounds located in step (a) and the positions of the corresponding QRS complexes or the corresponding first heart sounds;
(c) computing the median systolic length for the plurality of beats; and
(d) determining the position of the second heart sound for a beat by adding the median systolic length computed in step (c) to the position of the QRS complex or first heart sound corresponding to that beat.

18. The method of claim 11, wherein the step of selecting one or more beats for analysis comprises:
segmenting the acoustic signal into regions of interest using information obtained from an EKG, wherein the regions of interest are systolic regions or diastolic regions; and
discarding as too noisy those beats wherein the peak amplitude of the acoustic signal within the region of interest is greater than the amplitude of either the first or second heart sound or both.

19. The method of claim 11, wherein the step of selecting one or more beats for analysis comprises:
segmenting the acoustic signal into regions of interest using information obtained from an EKG, wherein the regions of interest are systolic regions or diastolic regions;
discarding those beats deemed too noisy; and
retaining a subset of the remaining beats based on their length.

20. The method of claim 19, wherein the retaining step is biased in favor of retaining longer beats.

21. The method of claim 19, wherein the retaining step is biased in favor of retaining shorter beats.

22. The method of claim 19, wherein the retaining step comprises:
(a) computing the median and standard deviation of the lengths of the remaining beats;
(b) computing values for either an upper threshold, a lower threshold, or both, wherein the value of an upper threshold is computed by adding a first predetermined percentage of the standard deviation to the median length, and a value for the lower threshold is computed by subtracting a second predetermined percentage of the standard deviation from the median length; and
(c) retaining beats whose length is greater than the lower threshold or is less than the upper threshold or falls within the interval between the upper and lower thresholds.

23. The method of claim 22, wherein the first and second predetermined percentages are the same.

24. The method of claim 22, wherein the first and second predetermined percentages are different.

25. The method of claim 22, further comprising the steps of:
(d) computing values for either a second upper threshold, a second lower threshold, or both, wherein the value of the second upper threshold is computed by adding a third predetermined percentage of the standard deviation to the upper threshold computed in step (b), and a value for the second lower threshold is computed by subtracting a fourth predetermined percentage of the standard deviation from the lower threshold computed in step (b); and
(e) retaining beats whose length is greater than the second lower threshold or is less than the second upper threshold or falls within the interval between the second upper and second lower thresholds.

26. The method of claim 25, wherein the third and fourth predetermined percentages are the same.

27. The method of claim 25, wherein the third and fourth predetermined percentages are different.

28. The method of claim 11, wherein performing a time-frequency analysis comprises performing a time-frequency decomposition.

29. The method of claim 28, wherein the time-frequency decomposition is performed by subjecting the acoustic signal to analysis by a filter bank comprising a plurality of frequency filters that separate the acoustic signal into its components within a plurality of frequency bands.

30. The method of claim 28, wherein the frequency filters have sufficient sharpness so as to substantially eliminate overlap between the frequency bands that they span.

31. The method of claim 28, wherein the frequency filter for each frequency band has a transition band width significantly lower than the width of adjacent frequency bands such that the energy content of the signal passed by each frequency filter is substantially free of energy contributed by signal from neighboring frequency bands.

32. The method of claim 31, wherein the transition bands of the frequency filters have widths less than approximately 10% of the widths of their passbands and the passbands of adjacent frequency filters.

33. The method of claim 28, wherein the frequency filters implement a finite impulse response approximation to the infinite impulse response of an ideal filter.

34. The method of claim 11, wherein the time-frequency analysis separates the acoustic signal into its components within a plurality of initial frequency bands, further comprising the step of aggregating multiple adjacent members of the plurality of initial frequency bands so as to create a set of broader composite frequency bands.

35. The method of claim 34, wherein the initial frequency bands are normalized prior to aggregation.

36. The method of claim 34, further comprising the step of taking the absolute value of the amplitudes of the signal in each initial frequency band prior to performing aggregation of the initial frequency bands.

37. The method of claim 11, wherein the step of selecting a plurality of beats for analysis is performed automatically.

38. The method of claim 11, wherein the time-frequency analysis separates the acoustic signal into its components within a plurality of frequency bands, and wherein the combining step creates a prototypical beat by, for each frequency band, aligning a plurality of beats in time, identifying a median set of amplitudes at each time instant for each of the frequency bands, wherein the median set consists of a predetermined number of amplitudes; and computing the mean of each median set, thereby obtaining a time-frequency decomposition of a prototypical beat.

39. The method of claim 11, wherein the time-frequency analysis separates the acoustic signal into its components within a plurality of initial frequency bands, and wherein the processing step comprises identifying one or more points in the cardiac cycle at which the distribution of energy at in at least one of the frequency bands or in a composite frequency band created by aggregating multiple adjacent initial frequency bands differs from the distribution of energy expected to be present in a normal subject or determining whether such a point or points exist.

40. The method of claim 11, wherein the processing step comprises computing one or more time metrics, amplitude metrics, or both, that characterize the distribution of energy at one or more points in the cardiac cycle in at least one of the frequency bands.

41. The method of claim 11, wherein the processing step comprises applying band-specific thresholding to one or more frequency bands.

42. The method of claim 41, wherein the step of applying band-specific thresholding comprises:
(a) identifying the point of maximum signal amplitude within a region of interest for one or more of the frequency bands;
(b) calculating the earliest point in the region of interest where the signal amplitude first exceeds a predetermined percentage of the maximum signal amplitude for the one or more frequency bands;
(c) for each point calculated in step (b), computing the time interval between the point calculated in step (b) and a second time point;
(d) scaling the time interval(s) computed in step (c) by the length of the region of interest; and
(e) comparing the scaled time interval(s) computed in step (d) with a predetermined value, thereby determining whether to classify the acoustic signal as indicative of the existence or severity of a clinical condition.

43. The method of claim 42, wherein the predetermined value differs for different frequency bands.

44. The method of claim 42, wherein the region of interest is the second half of systole, and the second time point is the time point at which S2 occurs.

45. The method of claim 39, 40, 41, or 42, wherein the processing step comprises:
identifying each beat as normal or as indicative of the existence or severity of a clinical condition; and
concluding that the subject has the clinical condition if a predetermined percentage of the beats indicate the existence or severity of the condition.

46. The method of claim 39, 40, 41, or 42, wherein the system creates a prototypical beat, and wherein the analyzing step comprises identifying the prototypical beat as normal or as indicative of the existence or severity of a clinical condition.

47. The method of claim 11, further comprising the step of:
outputting the clinical conclusion or recommendation.

48. The method of claim 47, further comprising outputting one or more of the following together with the clinical conclusion or recommendation: a display of at least a portion of the acoustic signal, a display of an EKG tracing, a display of a frequency decomposed acoustic signal representing one or more beats or a prototypical beat, and an audio playback of one or more beats or a prototypical beat.

49. The method of claim 48, wherein any of the displays may be annotated or unannotated.

50. The method of claim 47, wherein the clinical conclusion or recommendation is selected from the group consisting of: whether a pathological condition exists, whether a benign condition exists, the likelihood that a pathological condition exists, the likelihood that a benign condition exists, the identity of a pathological or benign condition that the subject may have, the qualitative or quantitative severity of a clinical condition, whether to make a referral to a specialist, whether to perform an additional diagnostic study, and whether to initiate or alter therapy.

51. The method of claim 50, wherein the condition is mitral valve prolapse.

52. The method of claim 51, wherein the system determines whether the subject has mitral valve prolapse.

53. The method of claim 50 or 51, wherein the system identifies regurgitant and non-regurgitant mitral valve prolapse.

54. A method of performing automated auscultation of the cardiovascular system, the method comprising steps of:
(a) selecting a plurality of beats;
(b) constructing a prototypical beat by combining information from the plurality of beats;
(c) computing a metric that characterizes the prototypical beat; and
(d) classifying the prototypical beat as indicative of the presence or absence of a disease or condition, or of its severity, by comparing the metric computed in step (c) with a value for the metric characteristic of a normal subject.

55. The method of claim 54, wherein the information comprises a time-frequency decomposition of each of the plurality of beats.

56. The method of claim 54, wherein the metric reflects the distribution, relative distribution, or both of energy in one or more frequency bands for the prototypical beat.

57. A method of performing automated auscultation of the cardiovascular system, the method comprising steps of:
(a) selecting a plurality of beats;
(b) constructing a prototypical beat by combining information from the plurality of beats; and
(c) presenting the prototypical beat, a time-frequency decomposition of the prototypical beat, or both, to a user, wherein presenting the prototypical beat comprises displaying an image of the beat, playing a recording of the beat or an enhanced version thereof, or both.

58. A method of performing automated auscultation of the cardiovascular system, the method comprising steps of:
(a) selecting one or more beats for analysis, wherein each beat comprises an acoustic signal emanating from the cardiovascular system;
(b) performing a time-frequency analysis of beats selected for analysis so as to provide information regarding the distribution of energy, the relative distribution of energy, or both, over different frequency ranges at one or more points in the cardiac cycle; and
(c) presenting information derived at least in part from the acoustic signal, wherein the information comprises one or more items selected from the group consisting of: a visual or audio presentation of a prototypical beat, a display of the time-frequency decomposition of one or more beats or prototypical beats, and a playback of the acoustic signal at a reduced rate with preservation of frequency content, wherein any of the foregoing items may be annotated or unannotated.

59. A system for performing automated auscultation of the cardiovascular system, the system comprising:
(a) a beat selection component that selects one or more beats for analysis, wherein each beat comprises an acoustic signal emanating from the cardiovascular system;
(b) a time-frequency analysis component that provides information regarding the distribution of energy, the relative distribution of energy, or both, over different frequency ranges at one or more points in the cardiac cycle; and
(c) a diagnostic module that processes the information to as to reach a clinically relevant conclusion or recommendation; and
(d) a presentation component that presents some or all of the information in a manner that facilitates a mechanistically comprehensible explanation of the basis for the conclusion or recommendation or how the conclusion or recommendation was reached.

60. A system for performing automated auscultation of the cardiovascular system of a subject, the method comprising steps of:
(a) a beat selection component that selects one or more beats for analysis, wherein each beat comprises an acoustic signal emanating from the cardiovascular system;
(b) a time-frequency analysis component that performs a time-frequency analysis of beats selected for analysis so as to provide information regarding the distribution of energy, the relative distribution of energy, or both, over different frequency ranges at one or more points in the cardiac cycle;
(c) an aggregation component that combines information obtained from a plurality of the beats selected for analysis; and
(d) a diagnostic module that processes the information to reach a clinically relevant conclusion or recommendation.

61. The system of claim 59 or 60, wherein the beat selection component selects beats automatically.

62. The system of claim 59 or 60, wherein the time-frequency analysis component comprises a time-frequency decomposition component.

63. The system of claim 59, further comprising:
an aggregation component that combines information obtained from a plurality of the beats selected for analysis.

64. The system of claim 60 or 63, further comprising one or more sensors that acquire the acoustic signal, an EKG, or both.

65. The system of claim 60 or 63, further comprising an electronic stethoscope comprising one or more sensors that acquire the acoustic signal, the EKG, or both.

66. The system of either claim 64 or 65, wherein the acoustic signal, the EKG, or both are transmitted wirelessly to a computing device.

67. A system for assisting in the clinical evaluation of a subject's cardiovascular system comprising:
an apparatus for performing automated auscultation of the cardiovascular system of a subject, wherein the apparatus analyzes an acoustic signal emanating from the cardiovascular system and provides a clinically relevant conclusion or recommendation; and
at least one audio-visual diagnostic aid that presents information derived at least in part from the acoustic signal.

68. A system for assisting in the clinical evaluation of a subject's cardiovascular system comprising:
an apparatus for performing automated auscultation of the cardiovascular system of a subject; and
at least one audio-visual diagnostic aid that presents information derived at least in part from the acoustic signal, wherein the information comprises one or more items selected from the group consisting of: a visual or audio presentation of a prototypical beat, a display of the time-frequency decomposition of one or more beats or prototypical beats, and a playback of the acoustic signal at a reduced rate with preservation of frequency content, wherein any of the foregoing items may be annotated or unannotated.

69. The system of claim 67 or 68, wherein the apparatus for performing automated auscultation is as described in claim 59.

70. The system of claim 67, wherein the audio-visual diagnostic aid presents information that directly reflects the mechanistic basis upon which the apparatus reached the clinically relevant conclusion or recommendation.

71. The system of claim 67, wherein the audio-visual diagnostic aid displays and/or plays back a prototypical acoustic signal obtained by aggregating information from a plurality of the subject's heart beats.

72. The system of claim 71, wherein the prototypical acoustic signal is enhanced to emphasize features of diagnostic significance.

73. The system of claim 67, wherein the audio-visual diagnostic aid additionally displays at least a portion of an EKG obtained from the subject, which EKG may be annotated and may be aligned with a display of an acoustic signal.

74. The system of claim 67, wherein the audio-visual diagnostic aid provides playback of the acoustic signal at a reduced rate with preservation of frequency content.

75. The system of claim 67, wherein the apparatus performs a time-frequency decomposition of the acoustic signal, and wherein the audio-visual diagnostic aid displays the time-frequency decomposition by separately displaying frequency components in a plurality of different frequency ranges.
